(19) United States
(12) Patent Application Publication
(10) Pub. No.: US 2020/0095223 A1
(43) Pub. Date: Mar. 26, 2020

(54) NOVEL TRIAZOLETHIONE DERIVATIVES

(71) Applicants: Bayer Aktiengesellschaft, Leverkusen (DE); Bayer CropScience Aktiengesellschaft, Monheim am Rhein (DE)

(72) Inventors: Andreas GOERTZ, Dormagen (DE); Ruth MEISSNER, Leverkusen (DE); Ricarda MILLER, Duesseldorf (DE); Sebastien NAUD, Lyon (FR); David BERNIER, Lyon (FR); Pierre GENIX, Lyon (FR); Stephane BRUNET, St Andre de Corcy (FR); Philippe KENNEL, Biot (FR); Pierre-Yves COQUERON, Lyon (FR)

(21) Appl. No.: 16/483,925

(22) PCT Filed: Jan. 29, 2019
(86) PCT No.: PCT/EP2018/052054

§ 371 Date: Aug. 6, 2019

(30) Foreign Application Priority Data
Feb. 8, 2017 (EP) 17155156.7

(51) Int. Cl. C07D 401/06; A01N 43/653

(52) U.S. Cl. CPC C07D 401/06; A01N 43/653

(57) ABSTRACT

The present invention relates to novel triazole derivatives of formula (I-S)

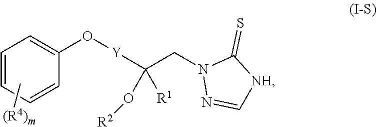

to processes for preparing these compounds, to compositions comprising these compounds, and to the use thereof as biologically active compounds, especially for control of harmful microorganisms in crop protection and in the protection of materials and as plant growth regulators.

The present invention relates to novel triazolethione derivatives, to processes for preparing these compounds, to compositions comprising these compounds, and to the use thereof as biologically active compounds, especially for control of harmful microorganisms in crop protection and in the protection of materials and as plant growth regulators.

It is already known that particular phenoxy-phenyl-substituted triazole derivatives show fungicidal efficacy (e.g. EP-A 0 275 955; J. Agric. Food Chem. 2009, 57, 4854-4860; CN-A 101225074, DE-A 40 03 180; EP-A 0 113 640; EP-A 0 470 466; U.S. Pat. No. 4,949,720; EP-A 0 126 430, DE-A 38 01 233; WO-A 2013/007767; WO-A 2013/010862; WO-A 2013/010885; WO-A 2013/010894; WO-A 2013/024075; WO-A 2013/024076; WO-A 2013/024077; WO-A 2013/024080; WO-A 2013/024081; WO-A 2013/024082; WO-A 2013/024083 and WO-A 2014/082872). It is also known that particular phenoxy-phenyl-substituted triazolethione derivatives (e.g. WO-A 2010/146114), particular hetaryloxy-phenyl-substituted triazolethione derivatives (e.g. WO-A 2010/146115) and particular phenoxy-hetaryl- and heterocyle-O-hetaryl-substituted triazolethione derivatives (e.g. WO-A 2010/146116) can be used in crop protection as fungicides.

Since the ecological and economic demands made on modern active ingredients, for example fungicides, are increasing constantly, for example with respect to efficacy, activity spectrum, toxicity, selectivity, application rate, formation of residues and favourable manufacture, and there can also be problems, for example, with resistances, there is a constant need to develop novel fungicidal compositions which have advantages over the known compositions at least in some areas.

In order to address this need, novel phenoxy-hetaryl-substituted triazole derivatives have been developed and are subject of WO-A 2017/029179. It now has been found that also the respective thione derivatives are useful fungicides.

Accordingly, the present invention provides novel triazole derivatives of formula (I-S)

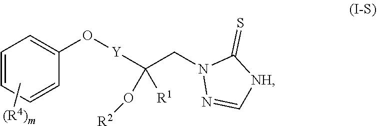

wherein
R¹ represents hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, phenyl, phenyl-C₁-C₄-alkyl, phenyl-C₂-C₄-alkenyl or phenyl-C₂-C₄-alkynyl;
R² represents hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, phenyl, phenyl-C₁-C₄-alkyl, phenyl-C₂-C₄-alkenyl or phenyl-C₂-C₄-alkynyl;
   wherein the aliphatic moieties, excluding cycloalkyl moieties, of R¹ and/or R² may carry 1, 2, 3 or up to the maximum possible number of identical or different groups R^{a} which independently of one another are selected from halogen, CN, nitro, phenyl, C₁-C₄-alkoxy and C₁-C₄-halogenalkoxy, wherein the phenyl may be substituted by 1, 2, 3, 4 or 5 substituents which independently of one another are selected from halogen, CN, nitro, C₁-C₄-alkyl, C₁-C₄-alkoxy, C₁-C₄-halogenalkyl, C₁-C₄-halogenalkoxy,
   and wherein the cycloalkyl and/or phenyl moieties of R¹ and/or R² may carry 1, 2, 3, 4, 5 or up to the maximum number of identical or different groups R^{b} which independently of one another are selected from halogen, CN, nitro, C₁-C₄-alkyl, C₁-C₄-alkoxy, C₁-C₄-halogenalkyl and C₁-C₄-halogenalkoxy;
   each R⁴ represents independently from each other halogen, CN, nitro, C₁-C₄-alkyl, C₁-C₄-halogenalkyl, C₃-C₆-cycloalkyl, C₃-C₆-halogencycloalkyl, C₁-C₄-alkyl-C₃-C₆-cycloalkyl, C₁-C₄-alkoxy, C₁-C₄-halogenalkoxy, hydroxy-substituted C₁-C₄-alkyl, C₂-C₆-alkenyl, C₂-C₆-halogenalkenyl, C₂-C₆-alkynyl, C₂-C₆-halogenalkynyl, C₁-C₄-alkylsulfanyl, C₁-C₄-halogenalkylsulfanyl, formyl, C₁-C₄-alkylcarbonyl, pentafluoro-λ⁶-sulfanyl or —C(R^{4a})═N—OR^{4b}, wherein R^{4a} and R^{4b} represent independently from each other hydrogen, C₁-C₆-alkyl or phenyl;
   m is an integer and is 0, 1, 2, 3, 4 or 5;
   Y represents a 6-membered aromatic heterocycle containing 1 or 2 nitrogen atom(s) as heteroatom(s) selected from

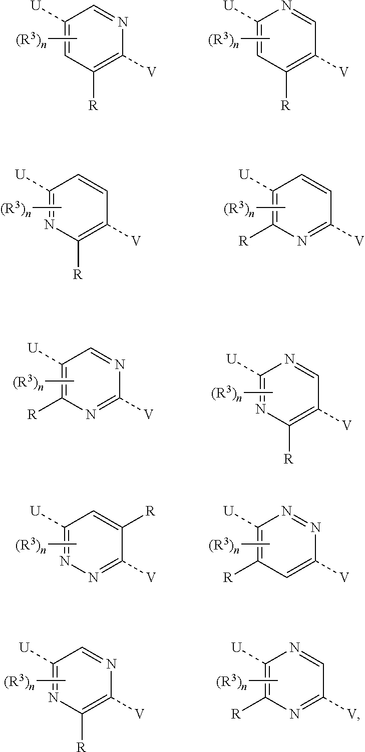

wherein Y is connected to the O of formula (I-S) via the bonds identified with “U” and Y is connected to the CR¹(OR²) moiety of formula (I-S) via the bonds identified with “V” and
wherein
R represents hydrogen, C₁-C₂-halogenalkyl, C₁-C₂-halogenalkoxy, C₁-C₂-alkylcarbonyl or halogen;
each R³ represents independently from each other halogen, CN, nitro, C₁-C₄-alkyl, C₁-C₄-halogenalkyl, C₁-C₄-alkoxy or C₁-C₄-halogenalkoxy;
n is an integer and is 0 or 1;

and its salts or N-oxides.

The salts or N-oxides of the triazole derivatives of formula (I-S) also have fungicidal properties.

The formula (I-S) provides a general definition of the triazole derivatives according to the invention. Preferred radical definitions for the formulae shown above and below are given below. These definitions apply to the end products of formula (I-S) and likewise to all intermediates.

The triazole derivatives of formula (I-S) can be present in any of their tautomeric forms, including the thiono forms according to formula

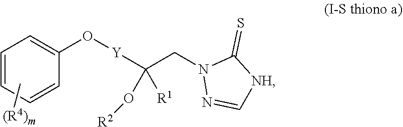

formula

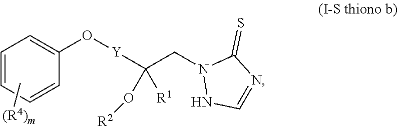

and the mercapto form according to formula

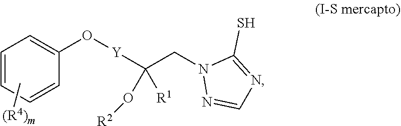

Herein, for reasons of simplicity only one thiono form is used in the formulae as drawn. However, triazole derivatives of formula (I-S) encompass all tautomeric forms, i.e. triazole derivatives of formula (I-S) in thiono form as drawn, as well as in the tautomeric thiono and mercapto forms. Same is true regarding any intermediate comprising the triazolethione ring.
R¹ preferably represents hydrogen, C₁-C₄-alkyl, C₂-C₆-alkenyl, C₂-C₆-alkynyl, cyclopropyl, phenyl, benzyl, phenylethenyl or phenylethinyl,
   wherein the aliphatic moieties, excluding the cycloalkyl moieties, of R¹ may carry 1, 2, 3 or up to the maximum possible number of identical or different groups R^{a} which independently of one another are selected from halogen, CN, nitro, phenyl, C₁-C₄-alkoxy and C₁-C₄-halogenalkoxy, wherein the phenyl may be substituted by 1, 2, 3, 4 or 5 substituents selected independently of one another from halogen, CN, nitro, C₁-C₄-alkyl, C₁-C₄-alkoxy, C₁-C₄-halogenalkyl, C₁-C₄-halogenalkoxy,
   and wherein the cycloalkyl and/or phenyl moieties of R¹ may carry 1, 2, 3, 4, 5 or up to the maximum number of identical or different groups R^{b} which independently of one another are selected from halogen, CN, nitro, C₁-C₄-alkyl, C₁-C₄-alkoxy, C₁-C₄-halogenalkyl and C₁-C₄-halogenalkoxy.
R¹ more preferably represents hydrogen, methyl, ethyl, propyl, isopropyl, butyl, cyclopropyl, benzyl, allyl, CH₂C≡C—CH₃ or CH₂C≡CH,
   wherein the aliphatic groups R¹ may carry 1, 2, 3 or up to the maximum possible number of identical or different groups R^{a} which independently of one another are selected from halogen, CN, nitro, phenyl, C₁-C₄-alkoxy and C₁-C₄-halogenalkoxy, wherein the phenyl may be substituted by 1, 2, 3, 4 or 5 substituents selected independently of one another from halogen, CN, nitro, C₁-C₄-alkyl, C₁-C₄-alkoxy, C₁-C₄-halogenalkyl, C₁-C₄-halogenalkoxy.
R¹ more preferably represents hydrogen, methyl, ethyl, propyl, isopropyl, butyl, cyclopropyl, CF₃, benzyl, allyl, CH₂C≡C—CH₃ or CH₂C≡CH.
R¹ more preferably represents hydrogen, methyl, ethyl or cyclopropyl.
R¹ more preferably represents hydrogen or methyl.
R¹ also more preferably represents methyl or cyclopropyl.
R¹ most preferably represents methyl.
R² preferably represents hydrogen, C₁-C₄-alkyl, allyl, propargyl or benzyl,
   wherein the aliphatic moieties of R² may carry 1, 2, 3 or up to the maximum possible number of identical or different groups R^{a} which independently of one another are selected from halogen, CN, nitro, phenyl, C₁-C₄-alkoxy and C₁-C₄-halogenalkoxy, wherein the phenyl may be substituted by 1, 2, 3, 4 or 5 substituents selected independently of one another from halogen, CN, nitro, C₁-C₄-alkyl, C₁-C₄-alkoxy, C₁-C₄-halogenalkyl, C₁-C₄-halogenalkoxy,
   and wherein the phenyl moieties of R² may carry 1, 2, 3, 4, 5 or up to the maximum number of identical or different groups R^{b} which independently of one another are selected from halogen, CN, nitro, C₁-C₄-alkyl, C₁-C₄-alkoxy, C₁-C₄-halogenalkyl and C₁-C₄-halogenalkoxy.
R² more preferably represents hydrogen, methyl, ethyl, isopropyl or allyl,
   wherein the aliphatic groups R² may carry 1, 2, 3 or up to the maximum possible number of identical or different groups R^{a} which independently of one another are selected from halogen, CN, nitro, phenyl, C₁-C₄-alkoxy and C₁-C₄-halogenalkoxy, wherein the phenyl may be substituted by 1, 2, 3, 4 or 5 substituents selected independently of one another from halogen, CN, nitro, C₁-C₄-alkyl, C₁-C₄-alkoxy, C₁-C₄-halogenalkyl, C₁-C₄-halogenalkoxy.
R² more preferably represents hydrogen or non-substituted methyl, ethyl, isopropyl or allyl.
R² more preferably represents hydrogen or methyl.
R² most preferably represents hydrogen.
Each R⁴ preferably represents independently from each other halogen, CN, nitro, C₁-C₄-alkyl, C₁-C₄-halogenalkyl, cyclopropyl, halogencyclopropyl, methylcyclopropyl, C₁-C₄-alkoxy, C₁-C₄-halogenalkoxy, C₂-C₆-alkenyl, C₂-C₆-halogenalkenyl, C₂-C₆-alkynyl, C₂-C₆-halogenalkynyl, C₁-C₄-alkylsulfanyl, C₁-C₄-halogenalkylsulfanyl, formyl, pentafluoro-λ⁶-sulfanyl or —C(R^{4a})═N—OR^{4b}, wherein R^{4a} and R^{4b} represent independently from each other hydrogen, C₁-C₆-alkyl or phenyl;
Each R⁴ more preferably represents independently from each other halogen, CN, nitro, C₁-C₄-alkyl, C₁-C₄-halogenalkyl, cyclopropyl, 1-fluorocyclopropyl, 1-chlorocyclopropyl, 1-methylcyclopropyl, C₁-C₄-alkoxy, C₁-C₄-halogenalkoxy, vinyl, allyl, propargyl, C₁-C₄-alkylsulfanyl, C₁-C₄-halogenalkylsulfanyl, formyl, pentafluoro-λ⁶-sulfanyl or —C(R^{4a})═N—OR^{4b},
   wherein R^{4a} and R^{4b} represent independently from each other hydrogen or C₁-C₄-alkyl, preferably hydrogen, methyl, ethyl, n-propyl, isopropyl, n-butyl, isobutyl or tert.-butyl, more preferably hydrogen or methyl.
Each R⁴ more preferably represents independently from each other Br, Cl, F, CN, nitro, methyl, ethyl, n-propyl, isopropyl, CHF₂, CF₃, cyclopropyl, 1-fluorocyclopropyl, 1-chlorocyclopropyl, 1-methylcyclopropyl, methoxy, OCF₃, vinyl, allyl, propargyl, SCH₃, SCF₃, formyl, pentafluoro-λ⁶-sulfanyl or —C(R^{4a})═N—OR^{4b},
   wherein R^{4a} and R^{4b} represent independently from each other hydrogen or C₁-C₄-alkyl, preferably hydrogen, methyl, ethyl, n-propyl, isopropyl, n-butyl, isobutyl or tert.-butyl, more preferably hydrogen or methyl.
Each R⁴ more preferably represents independently from each other Br, Cl, F, CN, nitro, methyl, ethyl, n-propyl, isopropyl, CHF₂, CF₃, methoxy, OCF₃, SCH₃, SCF₃, pentafluoro-λ⁶-sulfanyl or —C(R^{4a})═N—OR^{4b},
   wherein R^{4a} represents hydrogen, methyl, ethyl, n-propyl, isopropyl, n-butyl, isobutyl or tert.-butyl, preferably hydrogen or methyl, R^{4b} represents hydrogen, methyl, ethyl, n-propyl, isopropyl, n-butyl, isobutyl or tert.-butyl, preferably hydrogen or methyl.
Each R⁴ more preferably represents independently from each other CF₃, CHF₂, OCF₃, SCH₃, SCF₃, Br, Cl or pentafluoro-λ⁶-sulfanyl.
Each R⁴ more preferably represents independently from each other CF₃, CHF₂, OCF₃, Br, Cl or pentafluoro-λ⁶-sulfanyl.
Each R⁴ more preferably represents independently from each other CF₃, CHF₂, OCF₃, Br, Cl or pentafluoro-λ⁶-sulfanyl in the 4-position of the phenyl moiety of formula (I-S).
Each R⁴ most preferably represents independently from each other CF₃, Br or Cl in the 2- and/or 4-position, preferably the 4-position, of the phenyl moiety of formula (I-S).
m preferably is 0, 1, 2 or 3.
m more preferably is 0, 1 or 2.
m most preferably is 0 or 1.

In one preferred embodiment m is 0.

In another preferred embodiment m is 1 and R⁴ represents CF₃, CHF₂, OCF₃, Br, Cl or pentafluoro-λ⁶-sulfanyl, preferably CF₃, CHF₂, OCF₃, Br, Cl or pentafluoro-λ⁶-sulfanyl in the 4-position of the phenyl moiety of formula (I-S), more preferably CF₃, Br or Cl in the 2- and/or 4-position, preferably the 4-position, of the phenyl moiety of formula (I-S).
Y preferably represents

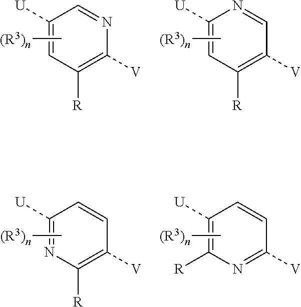

wherein R, R³ and n are defined as mentioned above for formula (I-S).
Y more preferably represents

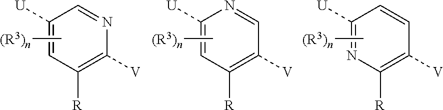

wherein R, R³ and n are defined as mentioned above for formula (I-S).
Y even more preferably represents

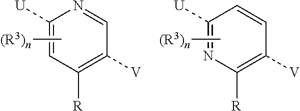

wherein R, R³ and n are defined as mentioned above for formula (I-S).
Y most preferably represents

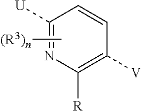

wherein R, R³ and n are defined as mentioned above for formula (I-S).
R preferably represents hydrogen, C₁-C₂-halogenalkyl or halogen;
R more preferably represents hydrogen, C₁-halogenalkyl, F or Cl.
R more preferably represents C₁-halogenalkyl, F or Cl.
R even more preferably represents CF₃ or Cl.
R most preferably represents CF₃.
n preferably is 0.

The radical definitions and explanations given above in general terms or stated within preferred ranges can, however, also be combined with one another as desired, i.e. including between the particular ranges and preferred ranges. They apply both to the end products and correspondingly to precursors and intermediates. In addition, individual definitions may not apply.

Preference is given to those compounds of the formula (I) in which each of the radicals have the abovementioned preferred definitions.

Particular preference is given to those compounds of the formula (I) in which each of the radicals have the abovementioned more and/or most preferred definitions.

In preferred embodiments of the present invention
R¹ represents hydrogen or C₁-C₄-alkyl, preferably hydrogen, methyl or ethyl;
R² represents hydrogen;
R⁴ represents CF₃, CHF₂, OCF₃, Br, Cl or pentafluoro-λ⁶-sulfanyl, preferably CF₃, CHF₂, OCF₃, Br, Cl or pentafluoro-λ⁶-sulfanyl in the 4-position of the phenyl moiety of formula (I-S), more preferably CF₃, Br or Cl in the 2- and/or 4-position, preferably the 4-position, of the phenyl moiety of formula (I-S);
m is 1;
Y represents

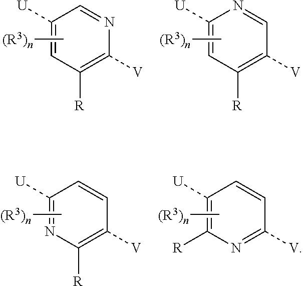

wherein Y is connected to the O of formula (I-S) via the bonds identified with “U” and Y is connected to the CR¹(OR²) moiety of formula (I-S) via the bonds identified with “V” and
wherein
R represents C₁-halogenalkyl, F or Cl, preferably CF₃ or Cl, more preferably CF₃; and
n is 0.

In further preferred embodiments of the present invention
R¹ represents hydrogen or C₁-C₄-alkyl, preferably hydrogen, methyl or ethyl;
R² represents hydrogen;
m is 0;
Y represents

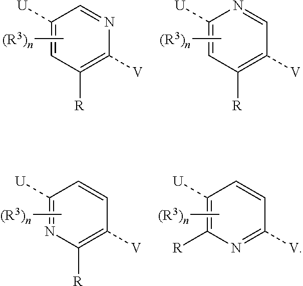

wherein Y is connected to the O of formula (I-S) via the bonds identified with “U” and Y is connected to the CR¹(OR²) moiety of formula (I-S) via the bonds identified with “V” and
wherein
R represents C₁-halogenalkyl, F or Cl, preferably CF₃ or Cl, more preferably CF₃; and
n is 0.

In more preferred embodiments of the present invention
R¹ represents hydrogen or methyl, preferably methyl;
R² represents hydrogen;
R⁴ represents CF₃, CHF₂, OCF₃, Br, Cl or pentafluoro-λ⁶-sulfanyl in the 4-position of the phenyl moiety of formula (I-S), preferably CF₃, Br or Cl in the 4-position of the phenyl moiety of formula (I-S);
m is 1;
Y represents

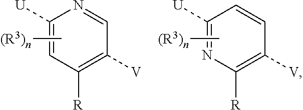

preferably

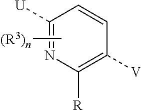

wherein Y is connected to the O of formula (I-S) via the bonds identified with “U” and Y is connected to the CR¹(OR²) moiety of formula (I-S) via the bonds identified with “V” and
wherein
R represents CF₃ or Cl, preferably CF₃; and
n is 0.

In further more preferred embodiments of the present invention
R¹ represents hydrogen or methyl, preferably methyl;
R² represents hydrogen;
m is 0;
Y represents

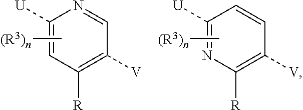

preferably

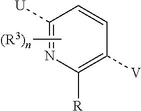

wherein Y is connected to the O of formula (I-S) via the bonds identified with “U” and Y is connected to the CR¹(OR²) moiety of formula (I-S) via the bonds identified with “V” and
wherein
R represents CF₃ or Cl, preferably CF₃; and
n is 0.

In the definitions of the symbols given in the above formulae, collective terms were used which are generally representative of the following substituents:

The definition C₁-C₆-alkyl comprises the largest range defined here for an alkyl radical. Specifically, this definition comprises the meanings methyl, ethyl, n-, isopropyl, n-, iso-, sec-, tert-butyl, and also in each case all isomeric pentyls and hexyls, such as methyl, ethyl, propyl, 1-methylethyl, butyl, 1-methylpropyl, 2-methylpropyl, 1,1-dimethylethyl, n-pentyl, 1-methylbutyl, 2-methylbutyl, 3-methylbutyl, 1,2-dimethylpropyl, 1,1-dimethylpropyl, 2,2-dimethylpropyl, 1-ethylpropyl, n-hexyl, 1-methylpentyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-3-methylpropyl, in particular propyl, 1-methylethyl, butyl, 1-methylbutyl, 2-methylbutyl, 3-methylbutyl, 1,1-dimethylethyl, 1,2-dimethylbutyl, 1,3-dimethylbutyl, n-pentyl, 1-methylbutyl, 1-ethylpropyl, hexyl, 3-methylpentyl. A preferred range is C₁-C₄-alkyl, such as methyl, ethyl, n-, isopropyl, n-, iso-, sec-, tert-butyl. The definition C₁-C₂-alkyl comprises methyl and ethyl.

The definition halogen comprises fluorine, chlorine, bromine and iodine. Halogen-substitution is generally indicated by the prefix halo, halogen or halogeno.

Halogen-substituted alkyl—e.g. referred to as halogenalkyl, halogenoalkyl or haloalkyl, e.g. C₁-C₄-halogenalkyl or C₁-C₂-halogenalkyl—represents, for example, C₁-C₄-alkyl or C₁-C₂-alkyl as defined above substituted by one or more halogen substituents which can be the same or different. Preferably C₁-C₄-halogenalkyl represents chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, 1,1-difluoroethyl, pentafluoroethyl, 1-fluoro-1-methylethyl, 2-fluoro-1,1-dimethylethyl, 2-fluoro-1-fluoromethyl-1-methylethyl, 2-fluoro-1,1-di(fluoromethyl)-ethyl, 1-chlorobutyl. Preferably C₁-C₂-halogenalkyl represents chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, 1,1-difluoroethyl, pentafluoroethyl.

Mono- or multiple fluorinated C₁-C₄-alkyl represents, for example, C₁-C₄-alkyl as defined above substituted by one or more fluorine substituent(s). Preferably mono- or multiple fluorinated C₁-C₄-alkyl represents fluoromethyl, difluoromethyl, trifluoromethyl, 1-fluoroethyl, 2-fluoroethyl, 2,2-difluoroethyl, 2,2,2-trifluoroethyl, pentafluoroethyl, 1-fluoro-1-methylethyl, 2-fluoro-1,1-dimethylethyl, 2-fluoro-1-fluoromethyl-1-methylethyl, 2-fluoro-1,1-di(fluoromethyl)-ethyl, 1-methyl-3-trifluoromethylbutyl, 3-methyl-1-trifluoromethylbutyl.

The definition C₂-C₆-alkenyl comprises the largest range defined here for an alkenyl radical. Specifically, this definition comprises the meanings ethenyl, n-, isopropenyl, n-, iso-, sec-, tert-butenyl, and also in each case all isomeric pentenyls, hexenyls, 1-methyl-1-propenyl, 1-ethyl-1-butenyl. Halogen-substituted alkenyl—e.g. referred to as C₂-C₆-haloalkenyl—represents, for example, C₂-C₆-alkenyl as defined above substituted by one or more halogen substituents which can be the same or different.

The definition C₂-C₆-alkynyl comprises the largest range defined here for an alkynyl radical. Specifically, this definition comprises the meanings ethynyl, n-, isopropynyl, n-, iso-, sec-, tert-butynyl, and also in each case all isomeric pentynyls, hexynyls. Halogen-substituted alkynyl—e.g. referred to as C₂-C₆-haloalkynyl—represents, for example, C₂-C₆-alkynyl as defined above substituted by one or more halogen substituents which can be the same or different.

The definition C₃-C₈-cycloalkyl comprises monocyclic saturated hydrocarbyl groups having 3 to 8 carbon ring members, such as cyclopropyl, cyclobutyl, cyclopentyl, cyclohexyl, cycloheptyl and cyclooctyl.

The definition halogen-substituted cycloalkyl—also referred to as halogenocycloalkyl, halocycloalkyl or halogencycloalkyl—comprises monocyclic saturated hydrocarbyl groups having 3 to 8 carbon ring members substituted by one or more halogen substituents which can be the same or different, such as 1-fluoro-cyclopropyl and 1-chloro-cyclopropyl.

The definition aryl comprises aromatic, mono-, bi- or tricyclic carbon rings, for example phenyl, naphthyl, anthracenyl (anthryl), phenanthracenyl (phenanthryl).

Optionally substituted radicals may be mono- or polysubstituted, where in the case of polysubstitution, the substituents may be identical or different.

Unless indicated otherwise, a group or a substituent which is substituted according to the invention preferably can be substituted by one or more group(s) selected from the list consisting of halogen, SH, nitro, hydroxyl, cyano, amino, sulfanyl, pentafluoro-λ⁶-sulfanyl, formyl, formyloxy, formylamino, carbamoyl, N-hydroxycarbamoyl, carbamate, (hydroxyimino)-C₁-C₆-alkyl, C₁-C₈-alkyl, C₁-C₈-halogenalkyl, C₁-C₈-alkyloxy, C₁-C₈-halogenalkyloxy, C₁-C₈-alkylthio, C₁-C₈-halogenalkylthio, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-halogenalkenyloxy, C₂-C₈-alkynyloxy, C₁-C₈-alkylamino, di-C₁-C₈-alkylamino, C₁-C₈-halogenalkylamino, di-C₁-C₈-halogenalkylamino, C₁-C₈-alkylaminoalkyl, di-C₁-C₈-alkylaminoalkyl, C₁-C₈-alkoxy, C₁-C₈-halogenoalkoxy, C₁-C₈-cyanoalkoxy, C₄-C₈-cycloalkylalkoxy, C₃-C₆-cycloalkoxy, C₂-C₈-alkoxyalkoxy, C₁-C₈-alkylcarbonylalkoxy, C₁-C₈-alkylsulfanyl, C₁-C₈-halogenoalkylsulfanyl, C₂-C₈-alkenyloxy, C₂-C₈-halogenoalkenyloxy, C₃-C₈-alkynyloxy, C₃-C₈-halogenoalkynyloxy, C₁-C₈-alkylcarbonyl, C₁-C₈-halogenoalkylcarbonyl, C₃-C₈-cycloalkylcarbonyl, C₃-C₈-halogenocycloalkylcarbonyl, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl, C₃-C₈-cycloalkoxycarbonyl, C₂-C₈-alkoxyalkylcarbonyl, C₂-C₈-halogenoalkoxyalkylcarbonyl, C₃-C₁₀-cycloalkoxyalkylcarbonyl, C₁-C₈-alkylaminocarbonyl, di-C₁-C₈-alkylaminocarbonyl, C₃-C₈-cycloalkylaminocarbonyl, C₁-C₈-alkylcarbonyloxy, C₁-C₈-halogenoalkylcarbonyloxy, C₃-C₈-cycloalkylcarbonyloxy, C₁-C₈-alkylcarbonylamino, C₁-C₈-halogenoalkylcarbonylamino, C₁-C₈-alkylaminocarbonyloxy, di-C₁-C₈-alkylaminocarbonyloxy, C₁-C₈-alkyloxycarbonyloxy, C₁-C₈-alkylsulfinyl, C₁-C₈-halogenoalkylsulfinyl, C₁-C₈-alkylsulfonyl, C₁-C₈-halogenoalkylsulfonyl, C₁-C₈-alkylaminosulfamoyl, di-C₁-C₈-alkylaminosulfamoyl, (C₁-C₈-alkoxyimino)-C₁-C₈-alkyl, (C₃-C₇-cycloalkoxyimino)-C₁-C₈-alkyl, hydroxyimino-C₁-C₈-alkyl, (C₁-C₈-alkoxyimino)-C₃-C₇-cycloalkyl, hydroxyimino-C₃-C₇-cycloalkyl, (C₁-C₈-alkylimino)-oxy, (C₁-C₈-alkylimino)-oxy-C₁-C₈-alkyl, (C₃-C₇-cycloalkylimino)-oxy-C₁-C₈-alkyl, (C₁-C₆-alkylimino)-oxy-C₃-C₇-cycloalkyl, (C₁-C₈-alkenyloxyimino)-C₁-C₈-alkyl, (C₁-C₈-alkynyloxyimino)-C₁-C₈-alkyl, 2-oxopyrrolidin-1-yl, (benzyloxyimino)-C₁-C₈-alkyl, C₁-C₈-alkoxyalkyl, C₁-C₈-alkylthioalkyl, C₁-C₈-alkoxyalkoxyalkyl, C₁-C₈-halogenoalkoxyalkyl, benzyl, phenyl, 5-membered heteroaryl, 6-membered heteroaryl, benzyloxy, phenyloxy, benzylsulfanyl, benzylamino, phenoxy, phenylsulfanyl, or phenylamino, wherein the benzyl, phenyl, 5-membered heteroaryl, 6-membered heteroaryl, 7-membered heteroaryl, benzyloxy or phenyloxy may be optionally substituted by one or more group(s) selected from the aforementioned list.

As not otherwise indicated—the definition 5-, 6- or 7-membered hetaryl or heteroaryl comprises unsaturated heterocyclic 5- to 7-membered ring containing up to 4 heteroatoms selected from N, O and S: for example 2-furyl, 3-furyl, 2-thienyl, 3-thienyl, 2-pyrrolyl, 3-pyrrolyl, 1-pyrrolyl, 3-pyrazolyl, 4-pyrazolyl, 5-pyrazolyl, 1-pyrazolyl, 1H-imidazol-2-yl, 1H-imidazol-4-yl, 1H-imidazol-5-yl, 1H-imidazol-1-yl, 2-oxazolyl, 4-oxazolyl, 5-oxazolyl, 2-thiazolyl, 4-thiazolyl, 5-thiazolyl, 3-isoxazolyl, 4-isoxazolyl, 5-isoxazolyl, 3-isothiazolyl, 4-isothiazolyl, 5-isothiazolyl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-3-yl, 1H-1,2,4-triazol-5-yl, 1H-1,2,4-triazol-1-yl, 4H-1,2,4-triazol-3-yl, 4H-1,2,4-triazol-4-yl, 1H-tetrazol-1-yl, 1H-tetrazol-5-yl, 2H-tetrazol-2-yl, 2H-tetrazol-5-yl, 1,2,4-oxadiazol-3-yl, 1,2,4-oxadiazol-5-yl, 1,2,4-thiadiazol-3-yl, 1,2,4-thiadiazol-5-yl, 1,3,4-oxadiazol-2-yl, 1,3,4-thiadiazol-2-yl, 1,2,3-oxadiazol-4-yl, 1,2,3-oxadiazol-5-yl, 1,2,3-thiadiazol-4-yl, 1,2,3-thiadiazol-5-yl, 1,2,5-oxadiazol-3-yl, 1,2,5-thiadiazol-3-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,4-triazin-6-yl.

If appropriate, the compounds according to the invention can be present as mixtures of different possible isomeric forms, in particular of stereoisomers, such as, for example, E and Z, threo and erythro, and also optical isomers, and, if appropriate, also of tautomers. What is claimed are both the E and the Z isomers, and also the threo and erythro, and the optical isomers, any mixtures of these isomers, and the possible tautomeric forms and any mixtures thereof.

If appropriate, the compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions) and to the mixtures of all the possible stereoisomers, in all proportions. The diastereoisomers and/or the optical isomers can be separated according to the methods which are known per se by the man ordinary skilled in the art.

If appropriate, the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

If appropriate, the compounds of the present invention can also exist in one or more geometric isomer forms depending on the relative position (syn/anti or cis/trans) of ring substituents. The invention thus relates equally to all syn/anti (or cis/trans) isomers and to all possible syn/anti (or cis/trans) mixtures, in all proportions. The syn/anti (or cis/trans) isomers can be separated according to general methods, which are known per se by the man ordinary skilled in the art.

Illustration of the processes and Intermediates

The present invention is furthermore related to processes for preparing compounds of formula (I-S).

The compounds of formula (I-S) can be obtained by various routes in analogy to prior art processes known (see e.g. DE-A 19744706, DE-A 19617282, DE-A 19528046, WO-A 2010/146032, WO-A 2011/113820, WO-A 2012/019981, WO-A 2012/041858) and by synthesis routes shown schematically below and in the experimental part of this application. Unless indicated otherwise, the radicals Y, R, R¹, R², R³, R⁴, m and n shown in the schemes below have the meanings given above for the compounds of formula (I-S). These definitions apply not only to the end products of the formula (I-S) but likewise to all intermediates. If individual compounds (I-S) cannot be obtained by those routes, they can be prepared by derivatization of other compounds (I-S).

Only for better understanding of the following Schemes the alcohols of formula (I-S) (R²═H) have been named as alcohols (I-S-H), although such alcohols (I-S-H) are encompassed by general formula (I-S) as defined above.

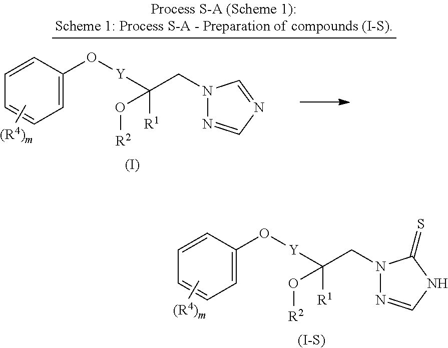

The triazole derivatives of formula (I-S) can be present in the mercapto form or in the tautomeric thiono forms. For reasons of simplicity only one thiono form is used for the compounds of formula (I-S) in Scheme 1.

The compounds (I) obtainable according to processes described below can be converted by means of methods described in the literature to the corresponding compounds (I-S) (see e.g. DE-A 19744706, DE-A 19617282, DE-A 19528046, WO-A 2010/146032, WO-A 2011/113820, WO-A 2012/019981, WO-A 2012/041858). Compounds of this general structure (I) are preferably reacted with bases, such as n-butyllithium and lithium diisopropylamide, or a Grignard reagent, such as isopropylmagnesium chloride, and subsequently with sulfur. Alternatively, compounds (I) are reacted with sulfur (DE-A 19744706) at elevated temperatures, preferentially in a solvent such as DMF.

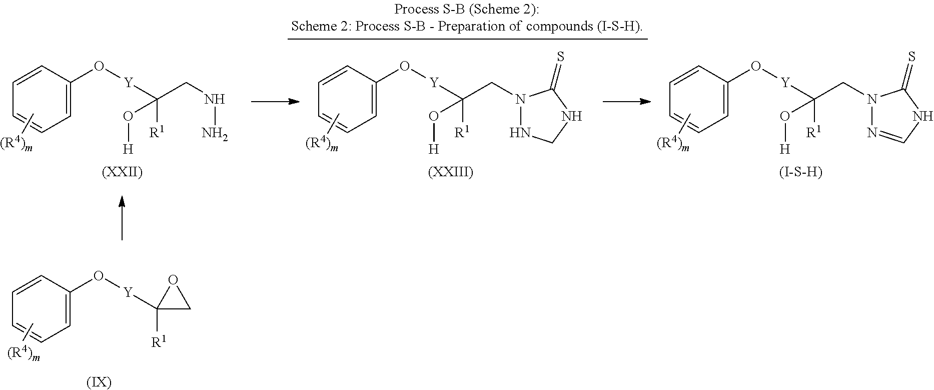

Compounds of formula (XXIII) and the triazole derivatives of formula (I-S-H) can be present in the mercapto form or in the tautomeric thiono forms. For reasons of simplicity only one thiono form is shown in Scheme 2.

Compounds (IX) obtainable according to the processes shown below can be converted by means of methods described in the literature to the corresponding compounds (I-S-H) (see e.g. WO-A 2001/46158 and references cited therein). Compounds of the general structure (IX) are preferably reacted with hydrazine hydrate to obtain compounds (XXII). These intermediates can be optionally obtained as salts or free bases, such as the corresponding hydrochlorides or the corresponding free hydrazines. By reacting compounds of general structure (XXII), optionally as salts or free bases, with formaldehyde and a thiocyanate salt, such as the sodium, potassium or ammonium salt, compounds (XXIII) can be obtained. Compounds (XXIII) can be optionally obtained as salts or free bases. The reaction can optionally be carried out in the presence of water. Compounds (XXIII) can be further oxidized to obtain compounds (I-S-H), preferentially in the presence of iron(III)-chloride and hydrochloric acid.

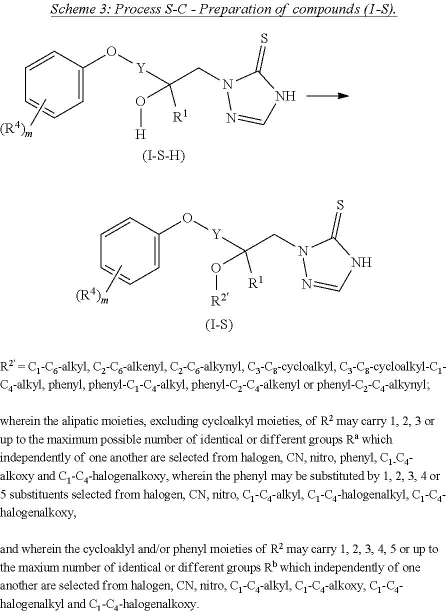

Compounds of formula (I-S-H) and the triazole derivatives of formula (I-S) can be present in the mercapto form or in the tautomeric thiono forms. For reasons of simplicity only one thiono form is shown in Scheme 3.

The compounds (I-S-H) obtainable according to process S-B can be converted by means of methods described in the literature to the corresponding compounds (I-S) (see e.g. DE-A 3202604, JP-A 02101067, EP-A 225739, CN-A 101824002, FR-A 2802772). Compounds of this general structure (I—S—H) are preferably reacted with alkylhalides, dialkylsulfates, anhydrides, acid chlorides, phosphorylchloride or alkylisocyanate, preferably in the presence of a base to obtain compounds (I-S).

Compounds of formula (I) can be obtained by various routes in analogy to prior art processes known (see e.g. J. Agric. Food Chem. (2009) 57, 4854-4860; EP-A 0 275 955; DE-A 40 03 180; EP-A 0 113 640; EP-A 0 126 430; WO-A 2013/007767 and references therein) and by synthesis routes shown schematically below and in the experimental part of this application. If individual compounds (I) cannot be obtained by those routes, they can be prepared by derivatization of other compounds (I).

Only for better understanding of the following Schemes the alcohols of formula (I) (R²═H) have been named as alcohols (I-H), although such alcohols (I-H) are encompassed by general formula (I) as defined above.

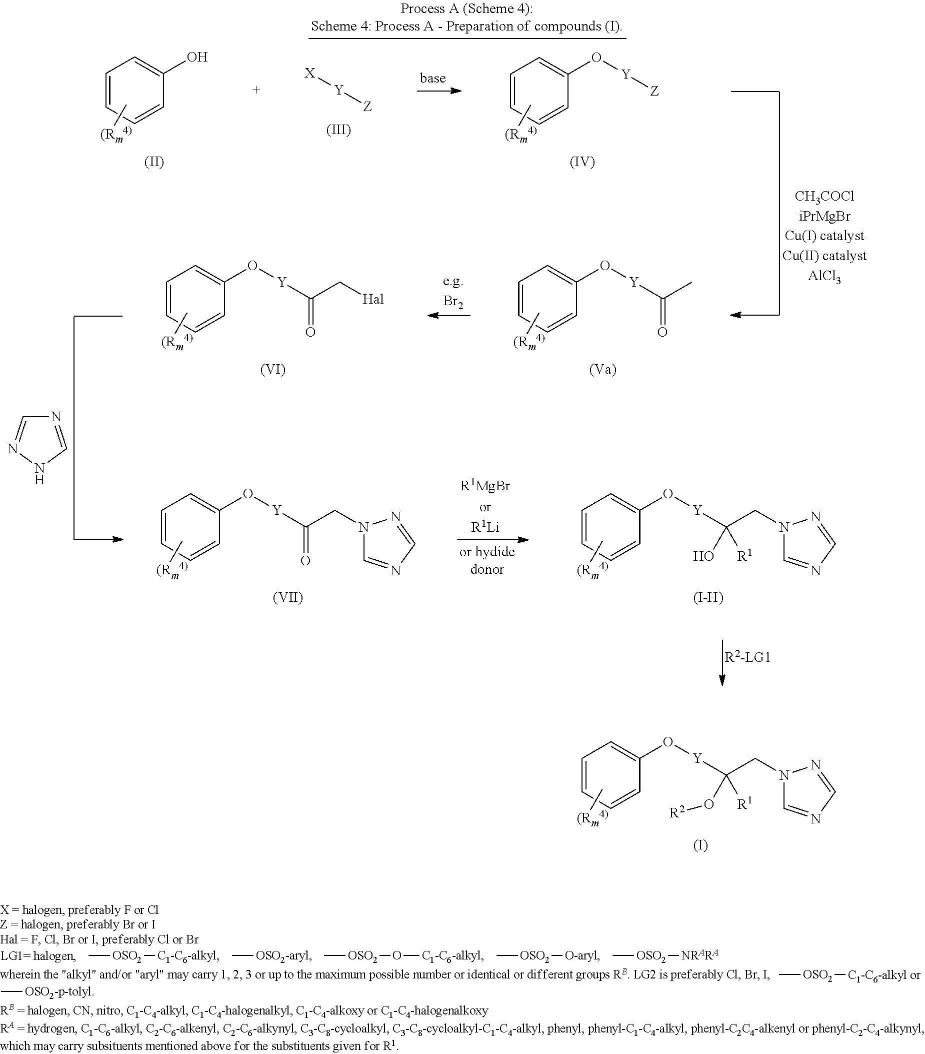

The compounds (II) and (III) (Scheme 1a) can be converted by means of methods described in the literature to the corresponding compounds (IV) and subsequently to compounds (Va), (VI), (VII), (I-H) and (I) (see WO-A 2013/007767). Phenols (II) are reacted with aryls (III), wherein X stands for F or C₁ and Z stands for Br or I. Z is in particular Br and the reaction is optionally performed in the presence of a base to obtain compounds (IV). These intermediates, in particular with Z being Br, are then transformed into Grignard reagents by the reaction with magnesium or by transmetallation reactions with reagents such as isopropylmagnesium halides and subsequently reacted with acetyl chloride to yield acetophenones (Va). Those reactions are preferably performed under anhydrous conditions and in the presence of a catalyst such as CuCl, CuCl₂, AlCl₃, LiCl and mixtures thereof. Compounds (Va) can be halogenated in a next step, for instance with Cl₂ or Br₂ in order to obtain α-haloketones (VI). The reactions are preferably carried out in an organic solvent such as diethyl ether, methyl tert.-butyl ether, methanol or acetic acid. The halogen in α-position, preferably Cl or Br, can be subsequently replaced by a 1,2,4-triazole. Preferably, this transformation is being conducted in the presence of a base, such as Na₂CO₃, K₂CO₃, Cs₂CO₃, NaOH, KOtBu, NaH or mixtures thereof, preferably in the presence of an organic solvent, such as tetrahydrofuran, dimethylformamide or toluene. Ketones (VII) are subsequently reacted with nucleophilic substrates, such as Grignard reagents R¹MgBr or organolithium compounds R¹Li or a hydride donor such as sodium borohydride to obtain alcohols (I-H). These transformations are preferably conducted under anhydrous conditions, optionally in the presence of a Lewis acid such as LaCl₃×2LiCl or MgBr₂×OEt₂. After further derivatization of alcohol (I-H) with an alkylating agent R²-LG compounds of the general formula (I) can be obtained. LG is a replaceable group such as halogen, alkylsulfonyl, alkylsulfonyloxy and arylsulfonyloxy, preferably Br, I and methylsulfonyloxy. These derivatizations are optionally performed in the presence of a base such as NaH and in the presence of an organic solvent such as tetrahydrofuran.

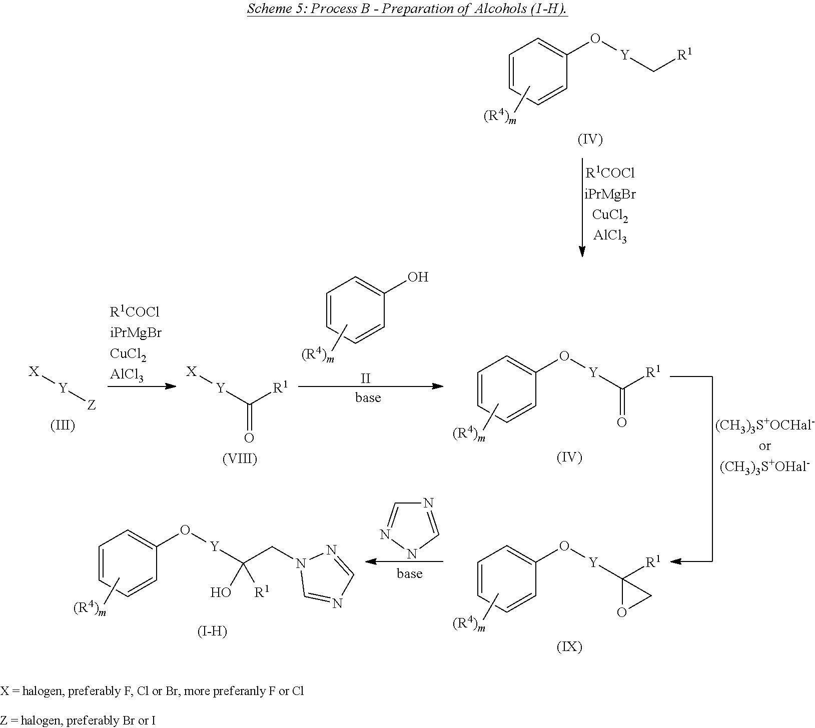

Compounds of the general structure (III), in particular with Z being Br, are being transformed into Grignard reagents by the reaction with magnesium or by transmetallation reactions with reagents such as isopropylmagnesium halides and subsequently reacted with acyl chlorides to yield ketones (VIII). Those reactions are preferably performed under anhydrous conditions and in the presence of a catalyst such as CuCl₂, AlCl₃, LiCl and mixtures thereof. Ketones (VIII) are subsequently reacted with Phenols (II), optionally in the presence of a base such as K₂CO₃ or Cs₂CO₃ and a solvent such as DMF (dimethylformamide), to obtain compounds (V). Alternatively, compounds (V) can be produced by the reaction of (IV) with magnesium or transmetallation reagents and subsequent reaction with acyl chlorides R¹COCl. Those reactions are preferably performed under anhydrous conditions and in the presence of a catalyst such as CuCl₂, AlCl₃, LiCl and mixtures thereof, Z being preferably Br. Thereafter, intermediates (V) can be converted by means of methods described in the literature to the corresponding epoxides (IX) (see e.g. EP-A 461 502, DE-A 33 15 681, EP-A 291 797, WO-A 2013/007767). Intermediates (V) are preferably reacted with trimethylsulfoxonium- or trimethylsulfonium-salts, which might be prepared in situ, preferably trimethylsulfoxonium halides, trimethylsulfonium halides, trimethylsulfoxonium methylsulfates or trimethylsulfonium methylsulfates, preferably in the presence of a base such as sodium hydroxide. Epoxides (IX) can be subsequently reacted with a 1,2,4-triazole in order to obtain compounds (I-H). Preferably, this transformation is being conducted in the presence of a base, such as Na₂CO₃, K₂CO₃, Cs₂CO₃, NaOH, KOtBu, NaH or mixtures thereof, preferably in the presence of an organic solvent, such as tetrahydrofuran, dimethylformamide or toluene.

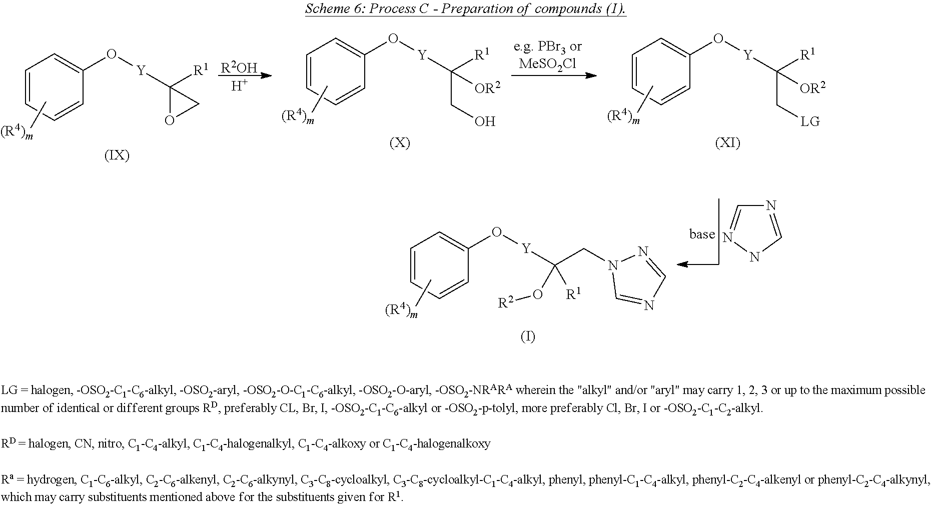

Epoxides of the general structure (IX) can be reacted with alcohols R²OH to yield alcohol (X). Preferentially, this transformation is being performed in the presence of an acid. Thereafter, alcohol (X) is being prepared for a nucleophilic substitution reaction. Along those lines, the alcohol functionality in compound (X) is being reacted with halogenating agents or sulfonating agents such as PBr₃, PCl₃, MeSO₂C₁, tosyl chloride or thionyl chloride to obtain compounds (XI). Subsequently, Intermediates (XI) can be reacted with a 1,2,4-triazole in order to obtain compounds (I). Optionally, this transformation is being conducted in the presence of a base, such as Na₂CO₃, K₂CO₃, Cs₂CO₃, NaOH, KOtBu, NaH or mixtures thereof, preferably in the presence of an organic solvent, such as tetrahydrofuran, dimethylformamide or toluene.

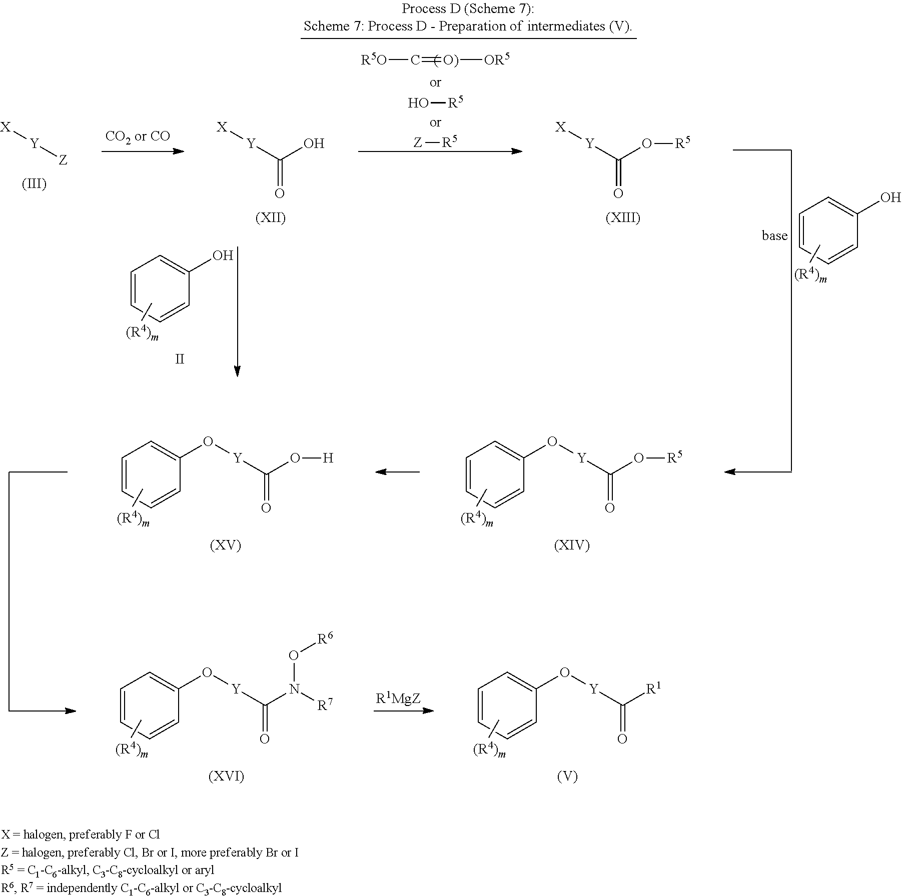

Compounds (III) can be converted by means of methods described in the literature to the corresponding compounds (XII) and subsequently to compounds (XIII), (XIV), (XV), (XVI) and (V). Alternatively, one or several reaction steps might be skipped. This is particularly true if certain protecting groups are not essential and thus process D might be shortened (e.g. (XII)→(XV)).

Compounds (III), wherein X stands for F or Cl and Z stands for Cl, Br or I, are optionally reacted with carbon dioxide or formate salts to obtain compounds (XII). This transformation is performed in the presence of reagents or catalysts such as lithium, magnesium, n-butyllithium, methyllithium or nickel (e.g. Organic & Biomolecular Chemistry, 8(7), 1688-1694; 2010; WO-A 2003/033504; Organometallics, 13(11), 4645-7; 1994 and references cited therein). Alternatively, compound (III) is reacted in a hydroxycarbonylation reaction with carbon monoxide or a formate salt, preferentially in the presence of a catalyst such as Pd(OAc)₂ and Co(OAc)₂ (e.g. Dalton Transactions, 40(29), 7632-7638; 2011; Synlett, (11), 1663-1666; 2006 and references cited therein).

Subsequently, acids (XII) are reacted with anhydrides R⁵O—C(═O)—OR⁵, alcohols HO—R⁵ or alkyl halides Z—R⁵ in order to obtain ester of the general structure (XIII) (e.g. Russian Journal of General Chemistry, 70 (9), 1371-1377, 2000; Bulletin of the Chemical Society of Japan 76 (8), 1645-1667, 2003). The reactions are preferentially performed in the presence of a coupling reagent such as CDI or DEAD and/or a base such as triethylamine or DMAP. Optionally, the corresponding acid chlorides are being formed before the reaction with alcohols HO—R⁵ takes place (e.g. WO-A 2007/059265). Ester (XIII) are subsequently reacted with Phenols (II), optionally in the presence of a base such as K₂CO₃, Cs₂CO₃, NEt3 or DABCO and a solvent such as DMF, to obtain compounds (XIV). The following hydrolysis can be carried out in the presence of an acid such as H₂SO₄, HNO₃ or p-toluenesulfonic acid or in the presence of a base such as KOH to yield acid (XV). Thereafter, acid (XV) can be reacted with alkoxyalkylamine, preferentially methoxymethylamine. The corresponding reaction can be carried out in the presence of reagents such as carbodiimides (e.g. WO-A 2011/076744), diimidazolyl ketone CDI, N-alkoxy-N-alkylcarbamoyl chlorides (e.g. Bulletin of the Korean Chemical Society 2002, 23, 521-524), S,S-di-2-pyridyl dithiocarbonates (e.g. Bulletin of the Korean Chemical Society 2001, 22, 421-423), trichloromethyl chloroformate (e.g. Synthetic communications 2003, 33, 4013-4018) or peptide coupling reagent HATU. Intermediates (V) can be obtained after reaction of Weinreb amides (XVI) with magnesium halides R₁MgZ such as methylmagnesium bromide, methylmagnesium chloride or ethylmagnesiumbromide, preferentially in a solvent such as THF.

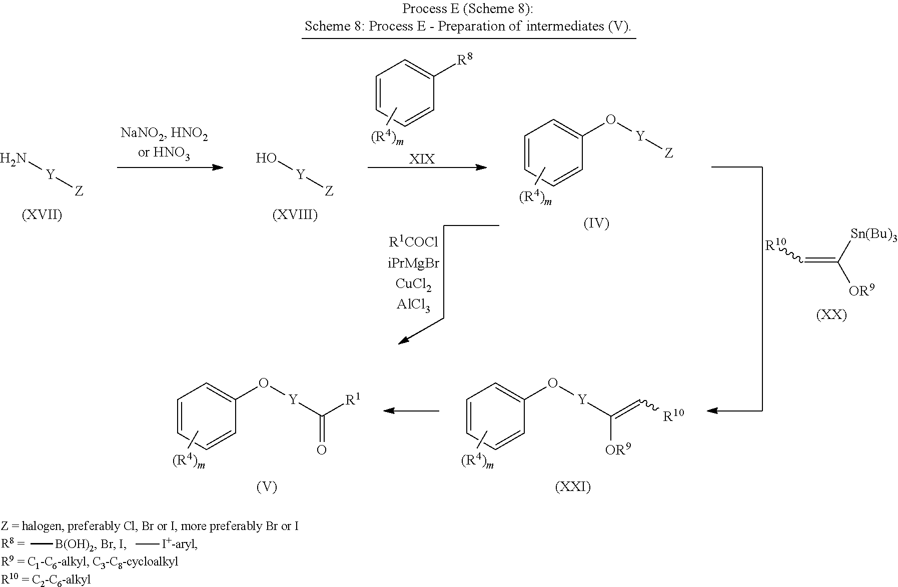

Amines (XVII) can be converted to the corresponding alcohols (XVIII) by means of methods described in the literature (e.g. Journal of Medicinal Chemistry 1999, 42, 95-108; WO-A 2007/017754; WO-A 2007/016525; Tetrahedron let. 2003, 44, 725-728), preferentially in the presence of sulfuric acid or hydrochloric acid as well as NaNO₂. Subsequently, alcohols (XVIII) can be converted to compounds of the general structure (IV) by literature know methods (e.g. Chemistry—A European Journal 2012, 18, 1414014149; Organic Letters 2011, 13, 1552-1555; Synlett 2012, 23, 101-106; WO-A 2005/040112; Organic Letters 2007, 9, 643-646; WO-A 2009/044160 and references cited therein). Compounds (XIX) could be for instance aryliodides which are optionally converted to diaryliodonium salts prior to the reaction, arylbromides or -iodides which are preferably reacted in the presence of a catalyst such as Cu or CuI or arylboronic acids or -esters which are preferentially reacted in the presence of a catalyst such as Cu(OAc)₂. Compounds (IV) can be reacted with a stannane such as (XX) in the presence of a transition metal catalyst such as Pd(PPh₃)₄, PdCl₂(PPh₃)₂, PdCl₂ or CuI (e.g. WO-A 2011/126960; WO-A 2011/088025; Journal of Organic Chemistry 1997, 62, 2774-2781; WO-A 2005/019212). Compounds (XXI) can be subsequently hydrolyzed to yield compounds (V), wherein R¹ is representd by C₁-C₆-alkyl, preferentially in the presence of an acid such as HCl or H₂SO₄ (e.g. Journal of Organic Chemistry 1990, 55, 3114-3118). Compounds (V) can be alternatively produced by the reaction of (IV) with magnesium or transmetallation reagents and subsequent reaction with acyl chlorides R¹COCl. Those reactions are preferably performed under anhydrous conditions and in the presence of a catalyst such as CuCl₂, AlCl₃, LiCl and mixtures thereof, Z being preferably Br.

General

The processes S-A to S-C and A to E for preparing compounds of the formula (I-S) and intermediates thereof are optionally performed using one or more reaction auxiliaries.

Useful reaction auxiliaries are, as appropriate, inorganic or organic bases or acid acceptors. These preferably include alkali metal or alkaline earth metal acetates, amides, carbonates, hydrogencarbonates, hydrides, hydroxides or alkoxides, for example sodium acetate, potassium acetate or calcium acetate, lithium amide, sodium amide, potassium amide or calcium amide, sodium carbonate, potassium carbonate or calcium carbonate, sodium hydrogencarbonate, potassium hydrogencarbonate or calcium hydrogencarbonate, lithium hydride, sodium hydride, potassium hydride or calcium hydride, lithium hydroxide, sodium hydroxide, potassium hydroxide or calcium hydroxide, n-butyllithium, sec-butyllithium, tert-butyllithium, lithium diisopropylamide, lithium bis(trimethylsilyl)amide, sodium methoxide, ethoxide, n- or i-propoxide, n-, i-, s- or t-butoxide or potassium methoxide, ethoxide, n- or i-propoxide, n-, i-, s- or t-butoxide; and also basic organic nitrogen compounds, for example trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]-non-5-ene (DBN) or 1,8-diazabicyclo[5.4.0]-undec-7-ene (DBU).

Useful reaction auxiliaries are, as appropriate, inorganic or organic acids. These preferably include inorganic acids, for example hydrogen fluoride, hydrogen chloride, hydrogen bromide and hydrogen iodide, sulphuric acid, phosphoric acid and nitric acid, and acidic salts such as NaHSO₄ and KHSO₄, or organic acids, for example, formic acid, carbonic acid and alkanoic acids such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, saturated or mono- or diunsaturated C₆-C₂₀ fatty acids, alkylsulphuric monoesters, alkylsulphonic acids (sulphonic acids having straight-chain or branched alkyl radicals having 1 to 20 carbon atoms), arylsulphonic acids or aryldisulphonic acids (aromatic radicals, such as phenyl and naphthyl, which bear one or two sulphonic acid groups), alkylphosphonic acids (phosphonic acids having straight-chain or branched alkyl radicals having 1 to 20 carbon atoms), arylphosphonic acids or aryldiphosphonic acids (aromatic radicals, such as phenyl and naphthyl, which bear one or two phosphonic acid radicals), where the alkyl and aryl radicals may bear further substituents, for example p-toluenesulphonic acid, salicylic acid, p-aminosalicylic acid, 2-phenoxybenzoic acid, 2-acetoxybenzoic acid, etc.

The processes S-A to S-C and A to E are optionally performed using one or more diluents. Useful diluents are virtually all inert organic solvents. Unless otherwise indicated for the above described processes S-A to S-C and A to E, these preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers such as diethyl ether, dibutyl ether and methyl tert-butyl ether, glycol dimethyl ether and diglycol dimethyl ether, tetrahydrofuran and dioxane, ketones such as acetone, methyl ethyl ketone, methyl isopropyl ketone and methyl isobutyl ketone, esters, such as methyl acetate and ethyl acetate, nitriles, for example acetonitrile and propionitrile, amides, for example dimethylformamide, dimethylacetamide and N-methylpyrrolidone, and also dimethyl sulphoxide, tetramethylenesulphone and hexamethylphosphoramide and DMPU.

In the processes the reaction temperatures can be varied within a relatively wide range. In general, the temperatures employed are between −78° C. and 250° C., preferably temperatures between −78° C. and 150° C.

The reaction time varies as a function of the scale of the reaction and of the reaction temperature, but is generally between a few minutes, e.g. 5 minutes, and 48 hours.

The processes are generally performed under standard pressure. However, it is also possible to work under elevated or reduced pressure.

For performance of the processes the starting materials required in each case are generally used in approximately equimolar amounts. However, it is also possible to use one of the components used in each case in a relatively large excess.

After a reaction has ended, the compounds are optionally separated from the reaction mixture by one of the customary separation techniques. If necessary, the compounds are purified by recrystallization or chromatography.

If appropriate, in the processes S-A to S-C and A to E according to the invention also salts and/or N-oxides of the starting compounds can be used.

The compounds of formula (I-S) and intermediates thereof can be converted into physiologically acceptable salts, e.g. acid addition salts or metal salt complexes.

Depending on the nature of the substituents defined above, the compounds of the formula (I-S) have acidic or basic properties and can form salts, if appropriate also inner salts, or adducts with inorganic or organic acids or with bases or with metal ions. If the compounds of the formula (I-S) carry amino, alkylamino or other groups which induce basic properties, these compounds can be reacted with acids to give salts, or they are directly obtained as salts in the synthesis. If the compounds of the formula (I-S) carries hydroxyl, carboxyl or other groups which induce acidic properties, these compounds can be reacted with bases to give salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl groups, mono-, di- and trialkanolamines of (C₁-C₄)-alkanols, choline and also chlorocholine.

The salts obtainable in this manner also have fungicidal properties.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, maleic acid, fumaric acid, tartaric acid, sorbic acid oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, 1,5-naphthalenedisulphonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others.

Particular preference is given to the metal ions of the elements of the fourth period. Here, the metals can be present in various valencies that they can assume.

The acid addition salts of the compounds of the formula (I-S) can be obtained in a simple manner by customary methods for forming salts, for example by dissolving a compound of the formula (I-S) in a suitable inert solvent and adding the acid, for example hydrochloric acid, and be isolated in a known manner, for example by filtration, and, if required, be purified by washing with an inert organic solvent.

Suitable anions of the salts are those which are preferably derived from the following acids: hydrohalic acids, such as, for example, hydrochloric acid and hydrobromic acid, furthermore phosphoric acid, nitric acid and sulphuric acid.

The metal salt complexes of compounds of the formula (I-S) can be obtained in a simple manner by customary processes, for example by dissolving the metal salt in alcohol, for example ethanol, and adding the solution to the compound of the formula (I-S). Metal salt complexes can be isolated in a known manner, for example by filtration, and, if required, be purified by recrystallization.

Salts of the intermediates can also be prepared according to the processes mentioned above for the salts of compounds of formula (I-S).

N-oxides of compounds of the formula (I-S) or intermediates thereof can be obtained in a simple manner by customary processes, for example by N-oxidation with hydrogen peroxide (H₂O₂), peracids, for example peroxy sulfuric acid or peroxy carboxylic acids, such as meta-chloroperoxybenzoic acid or peroxymonosulfuric acid (Caro's acid).

E.g. the corresponding N-oxides may be prepared starting from compounds (I-S) using conventional oxidation methods, e.g. by treating compounds (I-S) with an organic peracid such as metachloroperbenzoic acid (e.g. WO-A 2003/64572 or J. Med. Chem. 38 (11), 1892-1903, 1995); or with inorganic oxidizing agents such as hydrogen peroxide (e.g. J. Heterocyc. Chem. 18 (7), 1305-1308, 1981) or oxone (e.g. J. Am. Chem. Soc. 123 (25), 5962-5973, 2001). The oxidation may lead to pure mono-N-oxides or to a mixture of different N-oxides, which can be separated by conventional methods such as chromatography.

Composition/Formulation

The present invention further relates to a crop protection composition/formulation for controlling harmful microorganisms, especially unwanted fungi and bacteria, comprising an effective and non-phytotoxic amount of at least one inventive active ingredient. These are preferably fungicidal compositions which comprise agriculturally suitable auxiliaries, like solvents, carriers, surfactants or extenders.

In the context of the present invention, “control of harmful microorganisms” means a reduction in infestation by harmful microorganisms, compared with the untreated plant measured as fungicidal efficacy, preferably a reduction by 25-50%, compared with the untreated plant (100%), more preferably a reduction by 40-79%, compared with the untreated plant (100%); even more preferably, the infection by harmful microorganisms is entirely suppressed (by 70-100%). The control may be curative, i.e. for treatment of already infected plants, or protective, for protection of plants which have not yet been infected.

An “effective but non-phytotoxic amount” means an amount of the inventive composition which is sufficient to control the fungal disease of the plant in a satisfactory manner or to eradicate the fungal disease completely, and which, at the same time, does not cause any significant symptoms of phytotoxicity. In general, this application rate may vary within a relatively wide range. It depends on several factors, for example on the fungus to be controlled, the plant, the climatic conditions and the ingredients of the inventive compositions.

Suitable organic solvents include all polar and non-polar organic solvents usually employed for formulation purposes. Preferable the solvents are selected from ketones, e.g. methyl-isobutyl-ketone and cyclohexanone, amides, e.g. dimethyl formamide and alkanecarboxylic acid amides, e.g. N,N-dimethyl decaneamide and N,N-dimethyl octanamide, furthermore cyclic solvents, e.g. N-methyl-pyrrolidone, N-octyl-pyrrolidone, N-dodecyl-pyrrolidone, N-octyl-caprolactame, N-dodecyl-caprolactame and butyrolactone, furthermore strong polar solvents, e.g. dimethylsulfoxide, and aromatic hydrocarbons, e.g. xylol, Solvesso™, mineral oils, e.g. white spirit, petroleum, alkyl benzenes and spindle oil, also esters, e.g. propyleneglycol-monomethylether acetate, adipic acid dibutylester, acetic acid hexylester, acetic acid heptylester, citric acid tri-n-butylester and phthalic acid di-n-butylester, and also alkohols, e.g. benzyl alcohol and 1-methoxy-2-propanol.

According to the invention, a carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. Mixtures of such carriers can likewise be used.

Suitable solid filler and carrier include inorganic particles, e.g. carbonates, silikates, sulphates and oxides with an average particle size of between 0.005 and 20 μm, preferably of between 0.02 to 10 μm, for example ammonium sulphate, ammonium phosphate, urea, calcium carbonate, calcium sulphate, magnesium sulphate, magnesium oxide, aluminium oxide, silicium dioxide, so-called fine-particle silica, silica gels, natural or synthetic silicates, and alumosilicates and plant products like cereal flour, wood powder/sawdust and cellulose powder.

Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, maize cobs and tobacco stalks.

Useful liquefied gaseous extenders or carriers are those liquids which are gaseous at standard temperature and under standard pressure, for example aerosol propellants such as halohydrocarbons, and also butane, propane, nitrogen and carbon dioxide.

It is possible to further use tackifiers such as carboxymethylcellulose, and natural and synthetic polymers in the form of powders, granules or latices, such as gum arabic, polyvinyl alcohol and polyvinyl acetate, or else natural phospholipids, such as cephalins and lecithins, and synthetic phospholipids. Further additives may be mineral and vegetable oils.

If the extender used is water, it is also possible to employ, for example, organic solvents as auxiliary solvents. Useful liquid solvents are essentially: aromatics such as xylene, toluene or alkylnaphthalenes, chlorinated aromatics and chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

Suitable surfactants (adjuvants, emulsifiers, dispersants, protective colloids, wetting agent and adhesive) include all common ionic and non-ionic substances, for example ethoxylated nonylphenols, polyalkylene glycolether of linear or branched alcohols, reaction products of alkyl phenols with ethylene oxide and/or propylene oxide, reaction products of fatty acid amines with ethylene oxide and/or propylene oxide, furthermore fattic acid esters, alkyl sulfonates, alkyl sulphates, alkyl ethersulphates, alkyl etherphosphates, arylsulphate, ethoxylated arylalkylphenols, e.g. tristyryl-phenol-ethoxylates, furthermore ethoxylated and propoxylated arylalkylphenols like sulphated or phosphated arylalkylphenol-ethoxylates and -ethoxy- and -propoxylates. Further examples are natural and synthetic, water soluble polymers, e.g. lignosulphonates, gelatine, gum arabic, phospholipides, starch, hydrophobic modified starch and cellulose derivatives, in particular cellulose ester and cellulose ether, further polyvinyl alcohol, polyvinyl acetate, polyvinyl pyrrolidone, polyacrylic acid, polymethacrylic acid and co-polymerisates of (meth)acrylic acid and (meth)acrylic acid esters, and further co-polymerisates of methacrylic acid and methacrylic acid esters which are neutralized with alkalimetal hydroxide and also condensation products of optionally substituted naphthalene sulfonic acid salts with formaldehyde. The presence of a surfactant is necessary if one of the active ingredients and/or one of the inert carriers is insoluble in water and when application is effected in water. The proportion of surfactants is between 5 and 40 percent by weight of the inventive composition.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Antifoams which may be present in the formulations include e.g. silicone emulsions, longchain alcohols, fattiy acids and their salts as well as fluoroorganic substances and mixtures therof.

Examples of thickeners are polysaccharides, e.g. xanthan gum or veegum, silicates, e.g. attapulgite, bentonite as well as fine-particle silica.

If appropriate, it is also possible for other additional components to be present, for example protective colloids, binders, adhesives, thickeners, thixotropic substances, penetrants, stabilizers, sequestrants, complexing agents. In general, the active ingredients can be combined with any solid or liquid additive commonly used for formulation purposes.

The inventive active ingredients can be used as such or, depending on their particular physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, gas (under pressure), gas generating product, foams, pastes, pesticide coated seed, suspension concentrates, suspoemulsion concentrates, soluble concentrates, suspensions, wettable powders, soluble powders, dusts and granules, water-soluble and water-dispersible granules or tablets, water-soluble and water-dispersible powders for the treatment of seed, wettable powders, natural products and synthetic substances impregnated with active ingredient, and also microencapsulations in polymeric substances and in coating materials for seed, and also ULV cold-fogging and warm-fogging formulations.

Compositions according to the invention include not only formulations which are already ready for use and can be applied with a suitable apparatus to the plant or the seed, but also commercial concentrates which have to be diluted with water prior to use. Customary applications are for example dilution in water and subsequent spraying of the resulting spray liquor, application after dilution in oil, direct application without dilution, seed treatment or soil application of granules.

The inventive compositions and formulations generally contain between 0.05 and 99% by weight, 0.01 and 98% by weight, preferably between 0.1 and 95% by weight, more preferably between 0.5 and 90% of active ingredient, most preferably between 10 and 70% by weight. For special applications, e.g. for protection of wood and derived timber products the inventive compositions and formulations generally contain between 0.0001 and 95% by weight, preferably 0.001 to 60% by weight of active ingredient.

The contents of active ingredient in the application forms prepared from the commercial formulations may vary in a broad range. The concentration of the active ingredients in the application forms is generally between 0.000001 to 95% by weight, preferably between 0.0001 and 2% by weight.

The formulations mentioned can be prepared in a manner known per se, for example by mixing the active ingredients with at least one customary extender, solvent or diluent, adjuvant, emulsifier, dispersant, and/or binder or fixative, wetting agent, water repellent, if appropriate desiccants and UV stabilizers and, if appropriate, dyes and pigments, antifoams, preservatives, inorganic and organic thickeners, adhesives, gibberellins and also further processing auxiliaries and also water. Depending on the formulation type to be prepared further processing steps are necessary, e.g. wet grinding, dry grinding and granulation.

The inventive active ingredients may be present as such or in their (commercial) formulations and in the use forms prepared from these formulations as a mixture with other (known) active ingredients, such as insecticides, attractants, sterilants, bactericides, acaricides, nematicides, fungicides, growth regulators, herbicides, fertilizers, safeners and/or semiochemicals.

The inventive treatment of the plants and plant parts with the active ingredients or compositions is effected directly or by action on their surroundings, habitat or storage space by the customary treatment methods, for example by dipping, spraying, atomizing, irrigating, evaporating, dusting, fogging, broadcasting, foaming, painting, spreading-on, watering (drenching), drip irrigating and, in the case of propagation material, especially in the case of seeds, also by dry seed treatment, wet seed treatment, slurry treatment, incrustation, coating with one or more coats, etc. It is also possible to deploy the active ingredients by the ultra-low volume method or to inject the active ingredient preparation or the active ingredient itself into the soil.

Mixtures

Compounds of the formula (I-S) can be used as such or in compositions/formulations thereof and can be mixed with further known active ingredients, e.g. fungicides, bactericides, acaricides, nematicides or insecticides, in order thus to broaden, for example, the activity spectrum or to prevent development of resistance.

Useful mixing partners include, for example, known fungicides, insecticides, acaricides, nematicides or else bactericides (see also Pesticide Manual, 14th ed.).

A mixture with other known active ingredients, such as herbicides, or with fertilizers and growth regulators, safeners and/or semiochemicals, is also possible.

Hence, the invention further relates to mixtures and formulations, comprising at least one compound of formula (I-S) and at least a further active compound, preferably selected from fungicides, bactericides, acaricides, nematicides, insecticides, herbicides, fertilizers, growth regulators, safeners and/or semiochemicals, more preferably from fungicides, insecticides, herbicides, growth regulators and/or safeners, most preferably from fungicides.

Preferably the at least one further active compound is a fungicide selected from the following groups
(1) inhibitors of the ergosterol synthesis,
(2) inhibitors of the respiratory chain at complex I or II,
(3) inhibitors of the respiratory chain at complex III,
(4) inhibitors of the mitosis and cell division,
(5) compounds capable of having a multisite action,
(6) compounds capable of inducing a host defense,
(7) inhibitors of the amino acid and/or protein biosynthesis,
(8) inhibitors of the ATP production,
(9) inhibitors of the cell wall synthesis,
(10) inhibitors of the lipid and membrane synthesis,
(11) inhibitors of the melanine biosynthesis,
(12) inhibitors of the nucleic acid synthesis,
(13) inhibitors of the signal transduction,
(14) compounds capable of acting as uncoupler,
(15) other fungicides.

More preferably the at least one further active compound is selected from the group consisting of (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocycyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-)-1,2-oxazol-4-yl](pyridin-3-yl)methanol, (1.035) (S)-[3-(4-chloro-2-fluorophenyl)-5-(2,4-difluorophenyl)-)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Ipfentrifluconazole, (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-H-pyrazole-4-carboxamide, (2.031) 3-(difluoromethyl)-N-[(3R)-7-fluoro-,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034)N-(2-cyclopentyl-5-fluorobenzyl)-N-cyclopropyl-3-(difluoromethyl)-5-fluoro-1-methyl-1H-pyrazole-4-carboxamide, (2.035)N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037)N-(5-chloro-2-ethylbenzyl)-N-cyclopropyl-3-(difluoromethyl)-5-fluoro-1-methyl-1H-pyrazole-4-carboxamide, (2.038)N-(5-chloro-2-isopropylbenzyl)-N-cyclopropyl-3-(difluoromethyl)-5-fluoro-1-methyl-1H-pyrazole-4-carboxamide, (2.039)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041)N-[1-(2,4-dichlorophenyl)-1-methoxypropan-2-yl]-3-(difluoromethyl)-1-methyl-1H-pyrazole-4-carboxamide, (2.042)N-[2-chloro-6-(trifluoromethyl)benzyl]-N-cyclopropyl-3-(difluoromethyl)-5-fluoro-1-methyl-1H-pyrazole-4-carboxamide, (2.043)N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044)N-[5-chloro-2-(trifluoromethyl)benzyl]-N-cyclopropyl-3-(difluoromethyl)-5-fluoro-1-methyl-1H-pyrazole-4-carboxamide, (2.045)N-cyclopropyl-3-(difluoromethyl)-5-fluoro-1-methyl-N-[5-methyl-2-(trifluoromethyl)benzyl]-1H-pyrazole-4-carboxamide, (2.046)N-cyclopropyl-3-(difluoromethyl)-5-fluoro-N-(2-fluoro-6-isopropylbenzyl)-1-methyl-1H-pyrazole-4-carboxamide, (2.047)N-cyclopropyl-3-(difluoromethyl)-5-fluoro-N-(2-isopropyl-5-methylbenzyl)-1-methyl-1H-pyrazole-4-carboxamide, (2.048)N-cyclopropyl-3-(difluoromethyl)-5-fluoro-N-(2-isopropylbenzyl)-1-methyl-1H-pyrazole-4-carbothioamide, (2.049)N-cyclopropyl-3-(difluoromethyl)-5-fluoro-N-(2-isopropylbenzyl)-1-methyl-1H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-1H-pyrazole-4-carboxamide, (2.053)N-cyclopropyl-3-(difluoromethyl)-N-(2-ethyl-5-methylbenzyl)-5-fluoro-1-methyl-1H-pyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055)N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide, (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027)N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate, (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine. (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile, (6.001) acibenzolar-S-methyl, (6.002) isotianil, (6.003) probenazole, (6.004) tiadinil, (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinolone, (8.001) silthiofam, (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one, (10.001) propamocarb, (10.002) propamocarb hydrochloride, (10.003) tolclofos-methyl, (11.001) tricyclazole, (11.002) 2,2,2-trifluoroethyl {3-methyl-1-[(4-methylbenzoyl)amino]butan-2-yl}carbamate, (12.001) benalaxyl, (12.002) benalaxyl-M (kiralaxyl), (12.003) metalaxyl, (12.004) metalaxyl-M (mefenoxam), (13.001) fludioxonil, (13.002) iprodione, (13.003) procymidone, (13.004) proquinazid, (13.005) quinoxyfen, (13.006) vinclozolin, (14.001) fluazinam, (14.002) meptyldinocap, (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, and (15.062) 5-fluoro-4-imino-3-methyl-1-[(4-methylphenyl)sulfonyl]-3,4-dihydropyrimidin-2(1H)-one.

Plant/Crop Protection

The inventive active ingredients or compositions have potent microbicidal activity and can be used for control of unwanted microorganisms, such as fungi and bacteria, in crop protection and in the protection of materials.

The invention also relates to a method for controlling unwanted microorganisms, characterized in that the inventive active ingredients are applied to the phytopathogenic fungi, phytopathogenic bacteria and/or their habitat.

Fungicides can be used in crop protection for control of phytopathogenic fungi. They are characterized by an outstanding efficacy against a broad spectrum of phytopathogenic fungi, including soilborne pathogens, which are in particular members of the classes Plasmodiophoromycetes, Peronosporomycetes (Syn. Oomycetes), Chytridiomycetes, Zygomycetes, Ascomycetes, Basidiomycetes and Deuteromycetes (Syn. Fungi imperfecti). Some fungicides are systemically active and ca be used in plant protection as foliar, seed dressing or soil fungicide. Furthermore, they are suitable for combating fungi, which inter alia infest wood or roots of plant.

Bactericides can be used in crop protection for control of Pseudomonadaceae, Rhizobiaceae, Enterobacteriaceae, Corynebacteriaceae and Streptomycetaceae.

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi and Phakopsora meibomiae; Puccinia species, for example Puccinia recondite, P. triticina, P. graminis or P. striiformis; Uromyces species, for example Uromyces appendiculatus;

diseases caused by pathogens from the group of the Oomycetes, for example Albugo species, for example Algubo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidia form: Drechslera, Syn: Helminthosporium), Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans, Leptosphaeria nodorum; Magnaporthe species, for example Magnaporthe grisea; Microdochium species, for example Microdochium nivale; Mycosphaerella species, for example Mycosphaerella graminicola, M. arachidicola and M. fijiensis; Phaeosphaeria species, for example Phaeosphaeria nodorum; Pyrenophora species, for example Pyrenophora teres, Pyrenophora tritici repentis; Ramularia species, for example Ramularia collo-cygni, Ramularia areola; Rhynchosporium species, for example Rhynchosporium secalis; Septoria species, for example Septoria apii, Septoria lycopersii; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Rhizoctonia species, such as, for example Rhizoctonia solani; Sarocladium diseases caused for example by Sarocladium oryzae; Sclerotium diseases caused for example by Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Septoria species, for example Septoria nodorum;

diseases caused by smut fungi, for example Sphacelotheca species, for example Sphacelotheca reiliana; Tilletia species, for example Tilletia caries, T. controversa; Urocystis species, for example Urocystis occulta; Ustilago species, for example Ustilago nuda, U. nuda tritici;

fruit rot caused, for example, by Aspergillus species, for example Aspergillus flavus; Botrytis species, for example Botrytis cinerea; Penicillium species, for example Penicillium expansum and P. purpurogenum; Sclerotinia species, for example Sclerotinia sclerotiorum; Verticilium species, for example Verticilium alboatrum;

seed and soilborne decay, mould, wilt, rot and damping-off diseases caused, for example, by Alternaria species, caused for example by Alternaria brassicicola; Aphanomyces species, caused for example by Aphanomyces euteiches; Ascochyta species, caused for example by Ascochyta lentis; Aspergillus species, caused for example by Aspergillus flavus; Cladosporium species, caused for example by Cladosporium herbarum; Cochliobolus species, caused for example by Cochliobolus sativus; (Conidiaform: Drechslera, Bipolaris Syn: Helminthosporium); Colletotrichum species, caused for example by Colletotrichum coccodes; Fusarium species, caused for example by Fusarium culmorum; Gibberella species, caused for example by Gibberella zeae; Macrophomina species, caused for example by Macrophomina phaseolina; Monographella species, caused for example by Monographella nivalis; Penicillium species, caused for example by Penicillium expansum; Phoma species, caused for example by Phoma lingam; Phomopsis species, caused for example by Phomopsis sojae; Phytophthora species, caused for example by Phytophthora cactorum; Pyrenophora species, caused for example by Pyrenophora graminea; Pyricularia species, caused for example by Pyricularia oryzae; Pythium species, caused for example by Pythium ultimum; Rhizoctonia species, caused for example by Rhizoctonia solani; Rhizopus species, caused for example by Rhizopus oryzae; Sclerotium species, caused for example by Sclerotium rolfsii; Septoria species, caused for example by Septoria nodorum; Typhula species, caused for example by Typhula incarnata; Verticillium species, caused for example by Verticillium dahliae; cancers, galls and witches' broom caused, for example, by Nectria species, for example Nectria galligena; wilt diseases caused, for example, by Monilinia species, for example Monilinia laxa;

leaf blister or leaf curl diseases caused, for example, by Exobasidium species, for example Exobasidium vexans;

Taphrina species, for example Taphrina deformans;

decline diseases of wooden plants caused, for example, by Esca disease, caused for example by Phaemoniella clamydospora, Phaeoacremonium aleophilum and Fomitiporia mediterranea; Eutypa dyeback, caused for example by Eutypa lata; Ganoderma diseases caused for example by Ganoderma boninense; Rigidoporus diseases caused for example by Rigidoporus lignosus;

diseases of flowers and seeds caused, for example, by Botrytis species, for example Botrytis cinerea;

diseases of plant tubers caused, for example, by Rhizoctonia species, for example Rhizoctonia solani; Helminthosporium species, for example Helminthosporium solani;

Club root caused, for example, by Plasmodiophora species, for example Plamodiophora brassicae;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

The following diseases of soya beans can be controlled with preference:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The inventive fungicidal compositions can be used for curative or protective/preventive control of phytopathogenic fungi. The invention therefore also relates to curative and protective methods for controlling phytopathogenic fungi by the use of the inventive active ingredients or compositions, which are applied to the seed, the plant or plant parts, the fruit or the soil in which the plants grow.

The fact that the active ingredients are well tolerated by plants at the concentrations required for controlling plant diseases allows the treatment of above-ground parts of plants, of propagation stock and seeds, and of the soil.

According to the invention all plants and plant parts can be treated. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed muta-genesis, molecular or genetic markers or by bioengineering and genetic engineering methods. By plant parts is meant all above ground and below ground parts and organs of plants such as shoot, leaf, blossom and root, whereby for example leaves, needles, stems, branches, blossoms, fruiting bodies, fruits and seed as well as roots, corms and rhizomes are listed. Crops and vegetative and generative propagating material, for example cuttings, corms, rhizomes, runners and seeds also belong to plant parts.

The inventive active ingredients, when they are well tolerated by plants, have favourable homeotherm toxicity and are well tolerated by the environment, are suitable for protecting plants and plant organs, for enhancing harvest yields, for improving the quality of the harvested material. They can preferably be used as crop protection compositions. They are active against normally sensitive and resistant species and against all or some stages of development.

Plants which can be treated in accordance with the invention include the following main crop plants: maize, soya bean, alfalfa, cotton, sunflower, Brassica oil seeds such as Brassica napus (e.g. canola, rapeseed), Brassica rapa, B. juncea (e.g. (field) mustard) and Brassica carinata, Arecaceae sp. (e.g. oilpalm, coconut), rice, wheat, sugar beet, sugar cane, oats, rye, barley, millet and sorghum, triticale, flax, nuts, grapes and vine and various fruit and vegetables from various botanic taxa, e.g. Rosaceae sp. (e.g. pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g. olive tree), Actinidaceae sp., Lauraceae sp. (e.g. avocado, cinnamon, camphor), Musaceae sp. (e.g. banana trees and plantations), Rubiaceae sp. (e.g. coffee), Theaceae sp. (e.g. tea), Sterculiceae sp., Rutaceae sp. (e.g. lemons, oranges, mandarins and grapefruit); Solanaceae sp. (e.g. tomatoes, potatoes, peppers, capsicum, aubergines, tobacco), Liliaceae sp., Compositae sp. (e.g. lettuce, artichokes and chicory—including root chicory, endive or common chicory), Umbelliferae sp. (e.g. carrots, parsley, celery and celeriac), Cucurbitaceae sp. (e.g. cucumbers—including gherkins, pumpkins, watermelons, calabashes and melons), Alliaceae sp. (e.g. leeks and onions), Cruciferae sp. (e.g. white cabbage, red cabbage, broccoli, cauliflower, Brussels sprouts, pak choi, kohlrabi, radishes, horseradish, cress and chinese cabbage), Leguminosae sp. (e.g. peanuts, peas, lentils and beans—e.g. common beans and broad beans), Chenopodiaceae sp. (e.g. Swiss chard, fodder beet, spinach, beetroot), Linaceae sp. (e.g. hemp), Cannabeacea sp. (e.g. cannabis), Malvaceae sp. (e.g. okra, cocoa), Papaveraceae (e.g. poppy), Asparagaceae (e.g. asparagus); useful plants and ornamental plants in the garden and woods including turf, lawn, grass and Stevia rebaudiana; and in each case genetically modified types of these plants.

Plant Growth Regulation In some cases, the inventive compounds can, at particular concentrations or application rates, also be used as herbicides, safeners, growth regulators or agents to improve plant properties, or as microbicides, for example as fungicides, antimycotics, bactericides, viricides (including compositions against viroids) or as compositions against MLO (Mycoplasma-like organisms) and RLO (Rickettsia-like organisms). If appropriate, they can also be used as intermediates or precursors for the synthesis of other active ingredients.

The inventive active ingredients intervene in the metabolism of the plants and can therefore also be used as growth regulators.

Plant growth regulators may exert various effects on plants. The effect of the substances depends essentially on the time of application in relation to the developmental stage of the plant, and also on the amounts of active ingredient applied to the plants or their environment and on the type of application. In each case, growth regulators should have a particular desired effect on the crop plants.

Plant growth-regulating compounds can be used, for example, to inhibit the vegetative growth of the plants. Such inhibition of growth is of economic interest, for example, in the case of grasses, since it is thus possible to reduce the frequency of grass cutting in ornamental gardens, parks and sport facilities, on roadsides, at airports or in fruit crops. Also of significance is the inhibition of the growth of herbaceous and woody plants on roadsides and in the vicinity of pipelines or overhead cables, or quite generally in areas where vigorous plant growth is unwanted.

Also important is the use of growth regulators for inhibition of the longitudinal growth of cereal. This reduces or completely eliminates the risk of lodging of the plants prior to harvest. In addition, growth regulators in the case of cereals can strengthen the culm, which also counteracts lodging. The employment of growth regulators for shortening and strengthening culms allows the deployment of higher fertilizer volumes to increase the yield, without any risk of lodging of the cereal crop.

In many crop plants, inhibition of vegetative growth allows denser planting, and it is thus possible to achieve higher yields based on the soil surface. Another advantage of the smaller plants obtained in this way is that the crop is easier to cultivate and harvest.

Inhibition of the vegetative plant growth may also lead to enhanced yields because the nutrients and assimilates are of more benefit to flower and fruit formation than to the vegetative parts of the plants.

Frequently, growth regulators can also be used to promote vegetative growth. This is of great benefit when harvesting the vegetative plant parts. However, promoting vegetative growth may also promote generative growth in that more assimilates are formed, resulting in more or larger fruits.

In some cases, yield increases may be achieved by manipulating the metabolism of the plant, without any detectable changes in vegetative growth. In addition, growth regulators can be used to alter the composition of the plants, which in turn may result in an improvement in quality of the harvested products. For example, it is possible to increase the sugar content in sugar beet, sugar cane, pineapples and in citrus fruit, or to increase the protein content in soya or cereals. It is also possible, for example, to use growth regulators to inhibit the degradation of desirable ingredients, for example sugar in sugar beet or sugar cane, before or after harvest. It is also possible to positively influence the production or the elimination of secondary plant ingredients. One example is the stimulation of the flow of latex in rubber trees.

Under the influence of growth regulators, parthenocarpic fruits may be formed. In addition, it is possible to influence the sex of the flowers. It is also possible to produce sterile pollen, which is of great importance in the breeding and production of hybrid seed.

Use of growth regulators can control the branching of the plants. On the one hand, by breaking apical dominance, it is possible to promote the development of side shoots, which may be highly desirable particularly in the cultivation of ornamental plants, also in combination with an inhibition of growth. On the other hand, however, it is also possible to inhibit the growth of the side shoots. This effect is of particular interest, for example, in the cultivation of tobacco or in the cultivation of tomatoes.

Under the influence of growth regulators, the amount of leaves on the plants can be controlled such that defoliation of the plants is achieved at a desired time. Such defoliation plays a major role in the mechanical harvesting of cotton, but is also of interest for facilitating harvesting in other crops, for example in viticulture. Defoliation of the plants can also be undertaken to lower the transpiration of the plants before they are transplanted.

Growth regulators can likewise be used to regulate fruit dehiscence. On the one hand, it is possible to prevent premature fruit dehiscence. On the other hand, it is also possible to promote fruit dehiscence or even flower abortion to achieve a desired mass (“thinning”), in order to eliminate alternation. Alternation is understood to mean the characteristic of some fruit species, for endogenous reasons, to deliver very different yields from year to year. Finally, it is possible to use growth regulators at the time of harvest to reduce the forces required to detach the fruits, in order to allow mechanical harvesting or to facilitate manual harvesting.

Growth regulators can also be used to achieve faster or else delayed ripening of the harvested material before or after harvest. This is particularly advantageous as it allows optimal adjustment to the requirements of the market. Moreover, growth regulators in some cases can improve the fruit colour. In addition, growth regulators can also be used to concentrate maturation within a certain period of time. This establishes the prerequisites for complete mechanical or manual harvesting in a single operation, for example in the case of tobacco, tomatoes or coffee.

By using growth regulators, it is additionally possible to influence the resting of seed or buds of the plants, such that plants such as pineapple or ornamental plants in nurseries, for example, germinate, sprout or flower at a time when they are normally not inclined to do so. In areas where there is a risk of frost, it may be desirable to delay budding or germination of seeds with the aid of growth regulators, in order to avoid damage resulting from late frosts.

Finally, growth regulators can induce resistance of the plants to frost, drought or high salinity of the soil. This allows the cultivation of plants in regions which are normally unsuitable for this purpose.

Resistance Induction/Plant Health and Other Effects

The active compounds according to the invention also exhibit a potent strengthening effect in plants. Accordingly, they can be used for mobilizing the defences of the plant against attack by undesirable microorganisms.

Plant-strengthening (resistance-inducing) substances are to be understood as meaning, in the present context, those substances which are capable of stimulating the defence system of plants in such a way that the treated plants, when subsequently inoculated with undesirable microorganisms, develop a high degree of resistance to these microorganisms.

The active compounds according to the invention are also suitable for increasing the yield of crops. In addition, they show reduced toxicity and are well tolerated by plants.

Further, in context with the present invention plant physiology effects comprise the following: Abiotic stress tolerance, comprising temperature tolerance, drought tolerance and recovery after drought stress, water use efficiency (correlating to reduced water consumption), flood tolerance, ozone stress and UV tolerance, tolerance towards chemicals like heavy metals, salts, pesticides (safener) etc. . . . .

Biotic stress tolerance, comprising increased fungal resistance and increased resistance against nematodes, viruses and bacteria. In context with the present invention, biotic stress tolerance preferably comprises increased fungal resistance and increased resistance against nematodes

Increased plant vigor, comprising plant health/plant quality and seed vigor, reduced stand failure, improved appearance, increased recovery, improved greening effect and improved photosynthetic efficiency.

Effects on plant hormones and/or functional enzymes.

Effects on growth regulators (promoters), comprising earlier germination, better emergence, more developed root system and/or improved root growth, increased ability of tillering, more productive tillers, earlier flowering, increased plant height and/or biomass, shorting of stems, improvements in shoot growth, number of kernels/ear, number of ears/m², number of stolons and/or number of flowers, enhanced harvest index, bigger leaves, less dead basal leaves, improved phyllotaxy, earlier maturation/earlier fruit finish, homogenous riping, increased duration of grain filling, better fruit finish, bigger fruit/vegetable size, sprouting resistance and reduced lodging.

Increased yield, referring to total biomass per hectare, yield per hectare, kernel/fruit weight, seed size and/or hectolitre weight as well as to increased product quality, comprising:

improved processability relating to size distribution (kernel, fruit, etc.), homogenous riping, grain moisture, better milling, better vinification, better brewing, increased juice yield, harvestability, digestibility, sedimentation value, falling number, pod stability, storage stability, improved fiber length/strength/uniformity, increase of milk and/or meet quality of silage fed animals, adaption to cooking and frying;

further comprising improved marketability relating to improved fruit/grain quality, size distribution (kernel, fruit, etc.), increased storage/shelf-life, firmness/softness, taste (aroma, texture, etc.), grade (size, shape, number of berries, etc.), number of berries/fruits per bunch, crispness, freshness, coverage with wax, frequency of physiological disorders, colour, etc.;

further comprising increased desired ingredients such as e.g. protein content, fatty acids, oil content, oil quality, aminoacid composition, sugar content, acid content (pH), sugar/acid ratio (Brix), polyphenols, starch content, nutritional quality, gluten content/index, energy content, taste, etc.;

and further comprising decreased undesired ingredients such as e.g. less mycotoxines, less aflatoxines, geosmin level, phenolic aromas, lacchase, polyphenol oxidases and peroxidases, nitrate content etc.

Sustainable agriculture, comprising nutrient use efficiency, especially nitrogen (N)-use efficiency, phosphours (P)-use efficiency, water use efficiency, improved transpiration, respiration and/or CO₂ assimilation rate, better nodulation, improved Ca-metabolism etc. . . . .

Delayed senescence, comprising improvement of plant physiology which is manifested, for example, in a longer grain filling phase, leading to higher yield, a longer duration of green leaf colouration of the plant and thus comprising colour (greening), water content, dryness etc. . . . . Accordingly, in the context of the present invention, it has been found that the specific inventive application of the active compound combination makes it possible to prolong the green leaf area duration, which delays the maturation (senescence) of the plant. The main advantage to the farmer is a longer grain filling phase leading to higher yield. There is also an advantage to the farmer on the basis of greater flexibility in the harvesting time.

Therein “sedimentation value” is a measure for protein quality and describes according to Zeleny (Zeleny value) the degree of sedimentation of flour suspended in a lactic acid solution during a standard time interval. This is taken as a measure of the baking quality. Swelling of the gluten fraction of flour in lactic acid solution affects the rate of sedimentation of a flour suspension. Both a higher gluten content and a better gluten quality give rise to slower sedimentation and higher Zeleny test values. The sedimentation value of flour depends on the wheat protein composition and is mostly correlated to the protein content, the wheat hardness, and the volume of pan and hearth loaves. A stronger correlation between loaf volume and Zeleny sedimentation volume compared to SDS sedimentation volume could be due to the protein content influencing both the volume and Zeleny value (Czech J. Food Sci. Vol. 21, No. 3: 91-96, 2000).

Further the “falling number” as mentioned herein is a measure for the baking quality of cereals, especially of wheat. The falling number test indicates that sprout damage may have occurred. It means that changes to the physical properties of the starch portion of the wheat kernel has already happened. Therein, the falling number instrument analyzes viscosity by measuring the resistance of a flour and water paste to a falling plunger. The time (in seconds) for this to happen is known as the falling number. The falling number results are recorded as an index of enzyme activity in a wheat or flour sample and results are expressed in time as seconds. A high falling number (for example, above 300 seconds) indicates minimal enzyme activity and sound quality wheat or flour. A low falling number (for example, below 250 seconds) indicates substantial enzyme activity and sprout-damaged wheat or flour.

The term “more developed root system”/“improved root growth” refers to longer root system, deeper root growth, faster root growth, higher root dry/fresh weight, higher root volume, larger root surface area, bigger root diameter, higher root stability, more root branching, higher number of root hairs, and/or more root tips and can be measured by analyzing the root architecture with suitable methodologies and Image analysis programmes (e.g. WinRhizo).

The term “crop water use efficiency” refers technically to the mass of agriculture produce per unit water consumed and economically to the value of product(s) produced per unit water volume consumed and can e.g. be measured in terms of yield per ha, biomass of the plants, thousand-kernel mass, and the number of ears per m2.

The term “nitrogen-use efficiency” refers technically to the mass of agriculture produce per unit nitrogen consumed and economically to the value of product(s) produced per unit nitrogen consumed, reflecting uptake and utilization efficiency.

Improvement in greening/improved colour and improved photosynthetic efficiency as well as the delay of senescence can be measured with well-known techniques such as a HandyPea system (Hansatech). Fv/Fm is a parameter widely used to indicate the maximum quantum efficiency of photosystem II (PSII). This parameter is widely considered to be a selective indication of plant photosynthetic performance with healthy samples typically achieving a maximum Fv/Fm value of approx. 0.85. Values lower than this will be observed if a sample has been exposed to some type of biotic or abiotic stress factor which has reduced the capacity for photochemical quenching of energy within PSII. Fv/Fm is presented as a ratio of variable fluorescence (Fv) over the maximum fluorescence value (Fm). The Performance Index is essentially an indicator of sample vitality. (See e.g. Advanced Techniques in Soil Microbiology, 2007, 11, 319-341; Applied Soil Ecology, 2000, 15, 169-182.)

The improvement in greening/improved colour and improved photosynthetic efficiency as well as the delay of senescence can also be assessed by measurement of the net photosynthetic rate (Pn), measurement of the chlorophyll content, e.g. by the pigment extraction method of Ziegler and Ehle, measurement of the photochemical efficiency (Fv/Fm ratio), determination of shoot growth and final root and/or canopy biomass, determination of tiller density as well as of root mortality.

Within the context of the present invention preference is given to improving plant physiology effects which are selected from the group comprising: enhanced root growth/more developed root system, improved greening, improved water use efficiency (correlating to reduced water consumption), improved nutrient use efficiency, comprising especially improved nitrogen (N)-use efficiency, delayed senescence and enhanced yield.

Within the enhancement of yield preference is given as to an improvement in the sedimentation value and the falling number as well as to the improvement of the protein and sugar content—especially with plants selected from the group of cereals (preferably wheat).

Preferably the novel use of the fungicidal compositions of the present invention relates to a combined use of a) preventively and/or curatively controlling pathogenic fungi and/or nematodes, with or without resistance management, and b) at least one of enhanced root growth, improved greening, improved water use efficiency, delayed senescence and enhanced yield. From group b) enhancement of root system, water use efficiency and N-use efficiency is particularly preferred.

Seed Treatment

The invention further comprises a method for treating seed.

The invention further relates to seed which has been treated by one of the methods described in the previous paragraph. The inventive seeds are employed in methods for the protection of seed from harmful microorganisms. In these methods, seed treated with at least one inventive active ingredient is used.

The inventive active ingredients or compositions are also suitable for treating seed. A large part of the damage to crop plants caused by harmful organisms is triggered by the infection of the seed during storage or after sowing, and also during and after germination of the plant. This phase is particularly critical since the roots and shoots of the growing plant are particularly sensitive, and even minor damage may result in the death of the plant. There is therefore a great interest in protecting the seed and the germinating plant by using appropriate compositions.

The control of phytopathogenic fungi by treating the seed of plants has been known for a long time and is the subject of constant improvements. However, the treatment of seed entails a series of problems which cannot always be solved in a satisfactory manner. For instance, it is desirable to develop methods for protecting the seed and the germinating plant, which dispense with, or at least significantly reduce, the additional deployment of crop protection compositions after planting or after emergence of the plants. It is also desirable to optimize the amount of the active ingredient used so as to provide the best possible protection for the seed and the germinating plant from attack by phytopathogenic fungi, but without damaging the plant itself by the active ingredient employed. In particular, methods for the treatment of seed should also take account of the intrinsic fungicidal properties of transgenic plants in order to achieve optimal protection of the seed and the germinating plant with a minimum expenditure of crop protection compositions.

The present invention therefore also relates to a method for protection of seed and germinating plants from attack by phytopathogenic fungi, by treating the seed with an inventive composition. The invention likewise relates to the use of the inventive compositions for treatment of seed to protect the seed and the germinating plant from phytopathogenic fungi. The invention further relates to seed which has been treated with an inventive composition for protection from phytopathogenic fungi.

The control of phytopathogenic fungi which damage plants post-emergence is effected primarily by treating the soil and the above-ground parts of plants with crop protection compositions. Owing to the concerns regarding a possible influence of the crop protection compositions on the environment and the health of humans and animals, there are efforts to reduce the amount of active ingredients deployed.

One of the advantages of the present invention is that the particular systemic properties of the inventive active ingredients and compositions mean that treatment of the seed with these active ingredients and compositions not only protects the seed itself, but also the resulting plants after emergence, from phytopathogenic fungi. In this way, the immediate treatment of the crop at the time of sowing or shortly thereafter can be dispensed with.

It is likewise considered to be advantageous that the inventive active ingredients or compositions can especially also be used with transgenic seed, in which case the plant growing from this seed is capable of expressing a protein which acts against pests. By virtue of the treatment of such seed with the inventive active ingredients or compositions, merely the expression of the protein, for example an insecticidal protein, can control certain pests. Surprisingly, a further synergistic effect can be observed in this case, which additionally increases the effectiveness for protection against attack by pests.

The inventive compositions are suitable for protecting seed of any plant variety which is used in agriculture, in greenhouses, in forests or in horticulture and viticulture. In particular, this is the seed of cereals (such as wheat, barley, rye, triticale, sorghum/millet and oats), maize, cotton, soya beans, rice, potatoes, sunflower, bean, coffee, beet (for example sugar beet and fodder beet), peanut, oilseed rape, poppy, olive, coconut, cocoa, sugar cane, tobacco, vegetables (such as tomato, cucumbers, onions and lettuce), turf and ornamentals (see also below). The treatment of the seed of cereals (such as wheat, barley, rye, triticale and oats), maize and rice is of particular significance.

As also described below, the treatment of transgenic seed with the inventive active ingredients or compositions is of particular significance. This relates to the seed of plants containing at least one heterologous gene. Definition and examples of suitable heterologous genes are given below.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

When treating the seed, care must generally be taken that the amount of the inventive composition applied to the seed and/or the amount of further additives is selected such that the germination of the seed is not impaired, or that the resulting plant is not damaged. This has to be borne in mind in particular in the case of active ingredients which can have phytotoxic effects at certain application rates.

The inventive compositions can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. Nos. 4,272,417, 4,245,432, 4,808,430, 5,876,739, US 2003/0176428 A1, WO 2002/080675, WO 2002/028186.

The active ingredients usable in accordance with the invention can be converted to the customary seed dressing formulations, such as solutions, emulsions, suspensions, powders, foams, slurries or other coating compositions for seed, and also ULV formulations.

These formulations are prepared in a known manner, by mixing the active ingredients with customary additives, for example customary extenders and also solvents or diluents, dyes, wetting agents, dispersants, emulsifiers, antifoams, preservatives, secondary thickeners, adhesives, gibberellins and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of active agrochemical ingredients. Preference is given to using alkyl naphthalenesulphonates, such as diisopropyl or diisobutyl naphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ether, and the phosphated or sulphated derivatives thereof. Suitable anionic dispersants are especially lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylose.

The gibberellins which may be present in the seed dressing formulations usable in accordance with the invention may preferably be gibberellins A1, A3 (=gibberellic acid), A4 and A7; particular preference is given to using gibberellic acid. The gibberellins are known (cf. R. Wegler “Chemie der Pflanzenschutz-und Schidlingsbekimpfungsmittel” [Chemistry of the Crop Protection Compositions and Pesticides], vol. 2, Springer Verlag, 1970, p. 401-412).

The seed dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For treatment of seed with the seed dressing formulations usable in accordance with the invention, or the preparations prepared therefrom by adding water, all mixing units usable customarily for the seed dressing are useful. Specifically, the procedure in the seed dressing is to place the seed into a mixer, to add the particular desired amount of seed dressing formulations, either as such or after prior dilution with water, and to mix everything until the formulation is distributed homogeneously on the seed. If appropriate, this is followed by a drying process.

Mycotoxins

In addition, the inventive treatment can reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. clavifonne, P. roqueforti, Claviceps spec., such as C. purpurea, C. fusiformis, C. paspali, C. africana, Stachybotrys spec. and others.

Material Protection

The inventive active ingredients or compositions/formulations can also be used in the protection of materials, for protection of industrial materials against attack and destruction by harmful microorganisms, for example fungi and insects.

In addition, the inventive compounds can be used as antifouling compositions, alone or in combinations with other active ingredients.

Industrial materials in the present context are understood to mean inanimate materials which have been prepared for use in industry. For example, industrial materials which are to be protected by inventive active ingredients from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The inventive active ingredients or compositions may prevent adverse effects, such as rotting, decay, discoloration, decoloration or formation of mould.

In the case of treatment of wood the compounds/compositions according to the invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds according to the invention or a composition according to the invention; this includes for example direct application, spraying, dipping, injection or any other suitable means.

In addition, the inventive compounds can be used to protect objects which come into contact with saltwater or brackish water, especially hulls, screens, nets, buildings, moorings and signalling systems, from fouling.

The inventive method for controlling unwanted fungi can also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The inventive active ingredients may prevent adverse effects, such as rotting, decay, discoloration, decoloration or formation of mould.

Microorganisms capable of degrading or altering the industrial materials include, for example, bacteria, fungi, yeasts, algae and slime organisms. The inventive active ingredients preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Antimycotic Activity

In addition, the inventive active ingredients also have very good antimycotic activity. They have a very broad antimycotic activity spectrum, especially against dermatophytes and yeasts, moulds and diphasic fungi (for example against Candida species, such as C. albicans, C. glabrata), and Epidermophyton floccosum, Aspergillus species, such as A. niger and A. fumigatus, Trichophyton species, such as T. mentagrophytes, Microsporon species such as M. canis and M. audouinii. The list of these fungi by no means constitutes a restriction of the mycotic spectrum covered, and is merely of illustrative character.

The inventive active ingredients can therefore be used both in medical and in non-medical applications.

GMO

As already mentioned above, it is possible to treat all plants and their parts in accordance with the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts” or “parts of plants” or “plant parts” have been explained above. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA-miRNA—technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, intemode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance.

Plants or plant cultivars (obtained by plant biotechnology methods such as genetic engineering) which may also be treated according to the invention show altered quantity, quality and/or storage-stability of the harvested product and/or altered properties of specific ingredients of the harvested product.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants or plant cultivars (that can be obtained by plant biotechnology methods such as genetic engineering) which may also be treated according to the invention are plants, such as Tobacco plants, with altered post-translational protein modification patterns.

Application Rates and Timing

When using the inventive active ingredients as fungicides, the application rates can be varied within a relatively wide range, depending on the kind of application. The application rate of the inventive active ingredients is in the case of treatment of plant parts, for example leaves: from 0.1 to 10 000 g/ha, preferably from 10 to 1000 g/ha, more preferably from 10 to 800 g/ha, even more preferably from 50 to 300 g/ha (in the case of application by watering or dripping, it is even possible to reduce the application rate, especially when inert substrates such as rockwool or perlite are used);

in the case of seed treatment: from 2 to 200 g per 100 kg of seed, preferably from 3 to 150 g per 100 kg of seed, more preferably from 2.5 to 25 g per 100 kg of seed, even more preferably from 2.5 to 12.5 g per 100 kg of seed;

in the case of soil treatment: from 0.1 to 10 000 g/ha, preferably from 1 to 5000 g/ha.

These application rates are merely by way of example and are not limiting for the purposes of the invention.

The inventive active ingredients or compositions comprising such ingredient can thus be used to protect plants from attack by the pathogens mentioned for a certain period of time after treatment. The period for which protection is provided extends generally for 1 to 28 days, preferably for 1 to 14 days, more preferably for 1 to 10 days, most preferably for 1 to 7 days, after the treatment of the plants with the active ingredients, or for up to 200 days after a seed treatment.

The plants listed can particularly advantageously be treated in accordance with the invention with the compounds of the general formula (I-S) and the inventive compositions. The preferred ranges stated above for the active ingredients or compositions also apply to the treatment of these plants. Particular emphasis is given to the treatment of plants with the compounds or compositions specifically mentioned in the present text.

The invention is illustrated by the examples below. However, the invention is not limited to the examples.

### PREPARATION EXAMPLES

### Preparation of Compounds of the Formula (I-S) According to Process A-S

### Preparation of 2-[2-[6-(4-bromophenoxy)-2-(trifluoromethyl)-3-pyridyl]-2-hydroxy-propyl]-4H-1,2,4-triazole-3-thione (I-S-01) and 2-[2-hydroxy-2-[6-phenoxy-2-(trifluoromethyl)-3-pyridyl]propyl]-4H-1,2,4-triazole-3-thione (I-S-02)

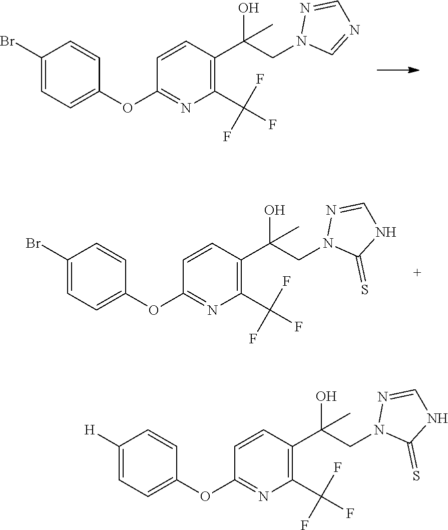 [SMILES: CC(O)(Cn1cncn1)c1ccc(Oc2ccc(Br)cc2)nc1C(F)(F)F ; CC(O)(Cn1nc[nH]c1=S)c1ccc(Oc2ccc(Br)cc2)nc1C(F)(F)F ; CC(O)(Cn1nc[nH]c1=S)c1ccc(Oc2ccccc2)nc1C(F)(F)F]

To a solution of 2-[6-(4-bromophenoxy)-2-(trifluoromethyl)pyridin-3-yl]-1-(1H-1,2,4-triazol-1-yl)propan-2-ol (600 mg, 1.35 mmol) in dry THF (tetrahydrofuran) (50 mL) at −78° C. was added a solution of n-butyllithium (1.19 mL, 2.97 mmol, 2.5 M in hexanes) drop wise, keeping the internal temperature below −70° C. The mixture was stirred for 1 hour (h) at −78° C., before sulfur (130 mg, 4.06 mmol) was added, and the resulting mixture stirred for another 2 h at −78° C., before the cooling bath was removed and the mixture was warmed to room temperature (rt, 21° C.) and stirred for another 1 h at rt. The reaction was then quenched with water, extracted with dichloromethane, washed with brine, dried over Na₂SO₄, and concentrated. Preparative HPLC yielded 50.5 mg (8% yield, 99% pure) of the target compound (I-S.01), and 84.5 mg (15% yield, 97% pure) of the target compound (I-S.02).

MS (ESI): 474.00 ([M−H]⁺) for (I-S.01)

MS (ESI): 397.09 ([M+H]+) for (I-S.02)

### Preparation of 2-[2-[6-(4-chlorophenoxy)-2-(trifluoromethyl)-3-pyridyl]-2-hydroxy-propyl]-4H-1,2,4-triazole-3-thione (I-S-03)

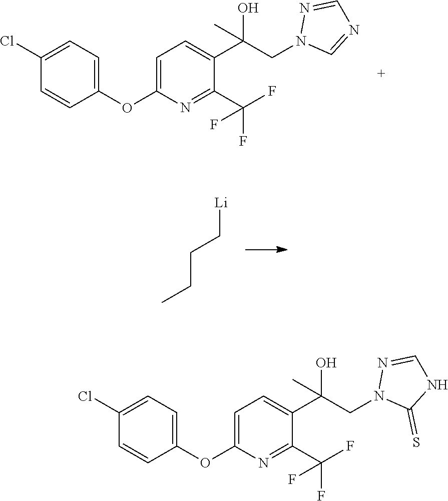

To a solution of 2-[6-(4-chlorophenoxy)-2-(trifluoromethyl)pyridin-3-yl]-1-(1H-1,2,4-triazol-1-yl)propan-2-ol (187 mg, 0.469 mmol) in dry THF (15 mL) at −78° C. was added a solution of n-butyllithium (0.413 mL, 1.03 mmol, 2.5 M in hexanes) drop wise, keeping the internal temperature below −70° C. The mixture was stirred for 1 hour (h) at −78° C., before sulfur (45.1 mg, 1.41 mmol) was added, and the resulting mixture stirred for another 2 h at −78° C., before the cooling bath was removed and the mixture was warmed to room temperature (rt) and stirred for another 1 h at rt. The reaction was then quenched with water, neutralized by addition of aqueous 0.1 N HCl, extracted with dichloromethane, washed with brine, dried over Na₂SO₄, and concentrated. Preparative HPLC yielded 167 mg (75% yield, 92% pure) of the target compound.

MS (ESI): 431.05 ([M+H]⁺)

### Preparation of 2-[2-[6-(4-bromophenoxy)-2-(trifluoromethyl)-3-pyridyl]-2-cyclopropyl-2-hydroxy-ethyl]-4H-1,2,4-triazole-3-thione (I-S-04) and 2-[2-cyclopropyl-2-hydroxy-2-[6-phenoxy-2-(trifluoromethyl)-3-pyridyl]ethyl]-4H-1,2,4-triazole-3-thione (I-S-06)

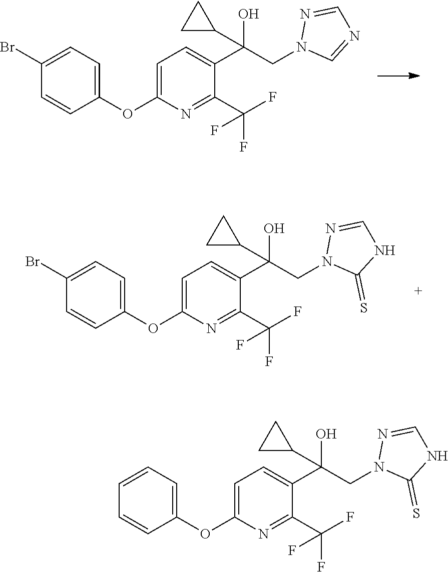 [SMILES: OC(Cn1cncn1)(c1ccc(Oc2ccc(Br)cc2)nc1C(F)(F)F)C1CC1 ; OC(Cn1nc[nH]c1=S)(c1ccc(Oc2ccc(Br)cc2)nc1C(F)(F)F)C1CC1 ; OC(Cn1nc[nH]c1=S)(c1ccc(Oc2ccccc2)nc1C(F)(F)F)C1CC1]

To a solution of 1-[6-(4-bromophenoxy)-2-(trifluoromethyl)-3-pyridyl]-1-cyclopropyl-2-(1,2,4-triazol-1-yl)ethanol (4.0 g, 8.52 mmol) in dry THF (320 mL) at −78° C. was added a solution of n-butyllithium (6.14 mL, 15.3 mmol, 2.5 M in hexanes) drop wise, keeping the internal temperature below −70° C. The mixture was stirred for 1 hour at −78° C., before sulfur (820 mg, 25.5 mmol) was added, and the resulting mixture stirred for another 1 h at −78° C., before the cooling bath was removed and the mixture was warmed to room temperature and stirred for another 1 h at rt. The reaction was then quenched with water, extracted with dichloromethane, washed with brine, dried over Na₂SO₄, and concentrated. Preparative HPLC yielded 480 mg (13% yield, 98% pure) of the target compound (I-S-06), and 258 mg (5.6% yield, 93% pure) of the target compound (I-S-04).

MS (ESI): 501.01 ([M]⁺) for (I-S-04)

MS (ESI): 422.10 ([M]⁺) for (I-S-06)

### Preparation of Compounds of the Formula (I) According to Process A

### Preparation of 2-[6-(4-chlorophenoxy)-2-(trifluoromethyl)pyridin-3-yl]-1-(1H-,2,4-triazol-1-yl)propan-2-ol

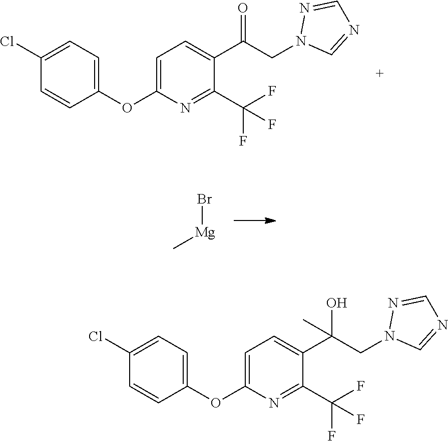

A solution of magnesium bromide diethyl etherate (4.8 g, 18.8 mmol) in dichloromethane (20 mL) and diethyl ether (10 mL) was cooled to 0° C., before a solution of 1-[6-(4-chlorophenoxy)-2-(trifluoromethyl)-3-pyridyl]-2-(1,2,4-triazol-1-yl)ethanone (1.80 g, 4.70 mmol) in dichloromethane (10 mL) was added and stirred for 30 min at 0° C. Then methylmagnesium bromide (3.1 mL, 9.4 mmol, 3M solution in ethyl ether) was added, the cooling bath was removed, and the mixture was stirred for 1.5 hours (h) at 21° C. (room temperature, rt), before the mixture was quenched with water, NH₄Cl (saturated aqueous solution), extracted with dichloromethane, dried (over MgSO₄), and concentrated. As the starting ketone and the target alcohol overlap in terms of retention time, the concentrated material (roughly 2 g of a thick colourless oil containing both ketone and alcohol) was dissolved in pyridine (15.0 mL), and treated with methoxylamine hydrochloride (313 mg, 3.75 mmol) at rt for 20 h (to convert the ketone into the corresponding methyl oxime, which has a significantly different retention time). The mixture was then diluted with dichloromethane, filtered over ChemElut, and concentrated. Preparative HPLC gave 319 mg (17% yield over two steps, 99% pure) of the target compound as colourless oil, which solidified upon standing.

MS (ESI): 398.08 ([M+H]⁺)

### Preparation of 2-[6-(4-chlorophenoxy)-4-(trifluoromethyl)-3-pyridyl]-1-(1,2,4-triazol-1-yl)propan-2-ol

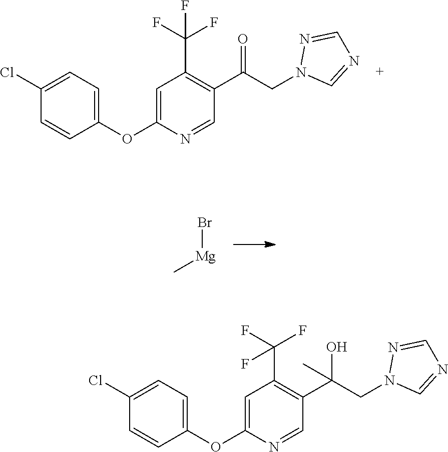

A solution of magnesium bromide diethyl etherate (1.2 g, 4.63 mmol) in dichloromethane (10 mL) was cooled to 0° C., before a solution of 1-[6-(4-chlorophenoxy)-4-(trifluoromethyl)-3-pyridyl]-2-(1,2,4-triazol-1-yl)ethanone (443 mg, 1.16 mmol) in dichloromethane (2 mL) was added and stirred for 30 min at 0° C. Then methylmagnesium bromide (0.78 mL, 2.3 mmol, 3M solution in ethyl ether) was added, the cooling bath was removed, and the mixture was stirred for 1 h at rt, before the mixture was quenched with water, extracted with dichloromethane, dried (over MgSO₄), and concentrated. Preparative HPLC gave 126.6 mg (27% yield, 100% pure) of the target compound as a colourless solid.

MS (ESI): 398.08 ([M+H]⁺)

### Preparation of 1-[6-(4-chlorophenoxy)-2-(trifluoromethyl)-3-pyridyl]-2-(1,2,4-triazol-1-yl)ethanol

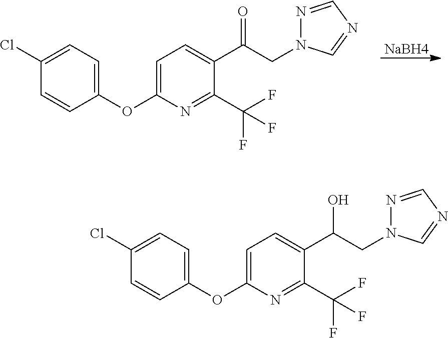

To a solution of 1-[6-(4-chlorophenoxy)-4-(trifluoromethyl)-3-pyridyl]-2-(1,2,4-triazol-1-yl)ethanone (800 mg, 1.67 mmol) in dry methanol (25.0 mL) at 5° C. was added sodium borohydride (127 mg, 3.3 mmol), the cooling bath was removed, mixture warmed to rt and stirred for 1 h. The mixture was then quenched with water, diluted with dichloromethane, filtered over ChemElut, and concentrated. Preparative HPLC gave 286 mg (60% yield, 100% pure) of the target compound as a colourless solid.

MS (ESI): 384.06 ([M+H]⁺)

### Preparation of 1-[6-(4-chlorophenoxy)-4-(trifluoromethyl)-3-pyridyl]-2-(1,2,4-triazol-1-yl)ethanol

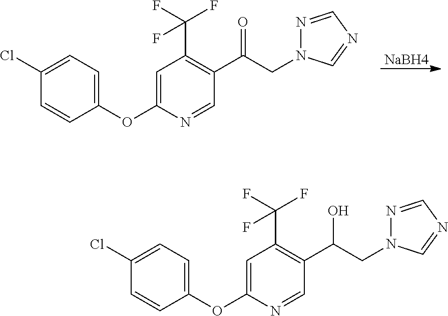

To a solution of 1-[6-(4-chlorophenoxy)-4-(trifluoromethyl)-3-pyridyl]-2-(1,2,4-triazol-1-yl)ethanone (518 mg, 1.35 mmol) in dry methanol (5.0 mL) at 5° C. was added sodium borohydride (102 mg, 2.7 mmol), the cooling bath was removed, mixture warmed to rt and stirred for 1 h. The mixture was then quenched with water, diluted with dichloromethane, filtered over ChemElut, and concentrated. Preparative HPLC gave 252 mg (48% yield, 100% pure) of the target compound as a colourless oil, which crystallized upon standing.

MS (ESI): 384.06 ([M+H]⁺)

### Preparation of Compounds of the Formula (VII) According to Process A

### Preparation of 1-[6-(4-chlorophenoxy)-2-(trifluoromethyl)-3-pyridyl]-2-(1,2,4-triazol-1-yl)ethanone

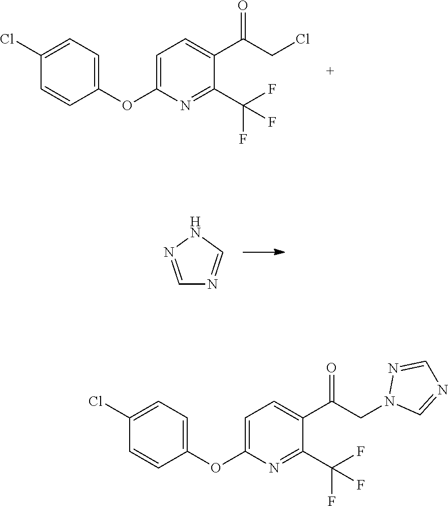 [SMILES: O=C(CCl)c1ccc(Oc2ccc(Cl)cc2)nc1C(F)(F)F ; c1nc[nH]n1 ; O=C(Cn1cncn1)c1ccc(Oc2ccc(Cl)cc2)nc1C(F)(F)F]

A mixture of 2-chloro-1-[6-(4-chlorophenoxy)-2-(trifluoromethyl)-3-pyridyl]ethanone (8.3 g, 23.7 mmol) and 1H-1,2,4-triazole (1.8 g, 26.0 mmol) in acetonitrile (80 mL) was heated to 75° C., before potassium carbonate (3.9 g, 28.5 mmol) was added. The heating was continued for 20 minutes (min) before the mixture was rapidly cooled to room temperature by addition of ice water, extracted with dichloromethane, dried (over MgSO₄), and concentrated. Flash column chromatography (gradient, up to DCM/10% MeOH in DCM=60/40, 254 nm) gave 4.30 g (43% yield, 91% pure) of the target compound as a yellow glass, which was used as such for the next reaction steps. A small quantity was further purified by HPLC to give the target product (100% pure) as a yellow solid.

MS (ESI): 382.04 ([M+H]⁺)

### Preparation of 1-[6-(4-chlorophenoxy)-4-(trifluoromethyl)-3-pyridyl]-2-(1,2,4-triazol-1-yl)ethanone

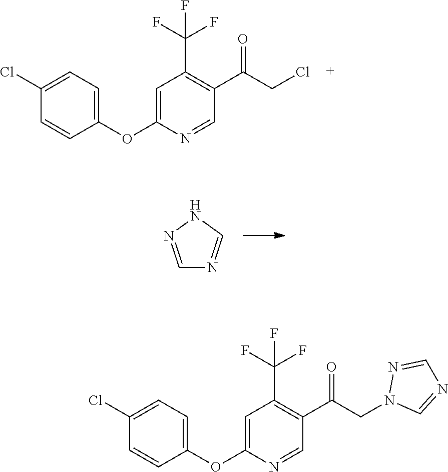 [SMILES: O=C(CCl)c1cnc(Oc2ccc(Cl)cc2)cc1C(F)(F)F ; c1nc[nH]n1 ; O=C(Cn1cncn1)c1cnc(Oc2ccc(Cl)cc2)cc1C(F)(F)F]

A mixture of 2-chloro-1-[6-(4-chlorophenoxy)-4-(trifluoromethyl)-3-pyridyl]ethanone (3.4 g, 9.71 mmol) and 1H-1,2,4-triazole (0.74 g, 10.6 mmol) in acetonitrile (50 mL) was heated to 75° C., before potassium carbonate (1.6 g, 11.6 mmol) was added. The heating was continued for 20 min before the mixture was rapidly cooled to room temperature by addition of ice water, extracted with dichloromethane, dried (over MgSO⁴), and concentrated. Flash column chromatography (gradient, up to DCM/10% MeOH in DCM=70/30, 254 nm) followed by preparative HPLC gave 1.50 g (40% yield, 100% pure) of the target compound as a yellow solid.

MS (ESI): 382.04 ([M+H]⁺)

### Preparation of Compounds of the Formula (VI) According to Process A

### Preparation of 2-chloro-1-[6-(4-chlorophenoxy)-2-(trifluoromethyl)-3-pyridyl]ethanone

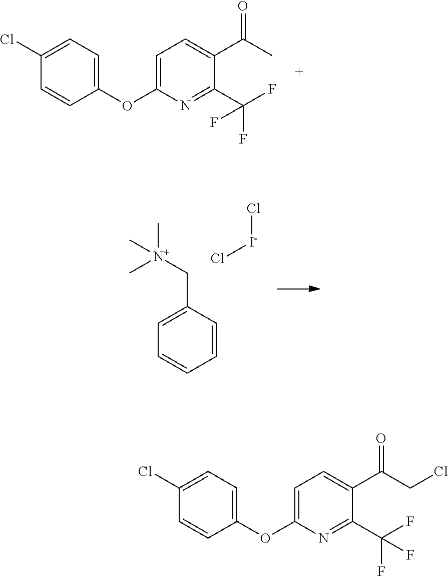

A mixture of 1-[6-(4-chlorophenoxy)-2-(trifluoromethyl)-3-pyridyl]ethanone (8.6 g, 27.2 mmol) and benzyltrimethylammonium dichloroiodate (18.9 g, 54.4 mmol) in 1,2-dichloroethane (60 mL) and methanol (20 mL) was heated to 75° C. for 4 h, before the mixture was concentrated, then diluted with ethyl acetate, washed with Na₂S₂O₃ (10% w/w aqueous solution), washed with brine, dried (over MgSO₄), concentrated, and passed over a plug of silica (heptane/ethyl acetate=1/1, 254 nm) to give 8.3 g (83% yield, 96% pure) of the target compound as a pale yellow solid.

MS (ESI): 348.99 ([M+H]⁺)

### Preparation of 2-chloro-1-[6-(4-chlorophenoxy)-4-(trifluoromethyl)-3-pyridyl]ethanone

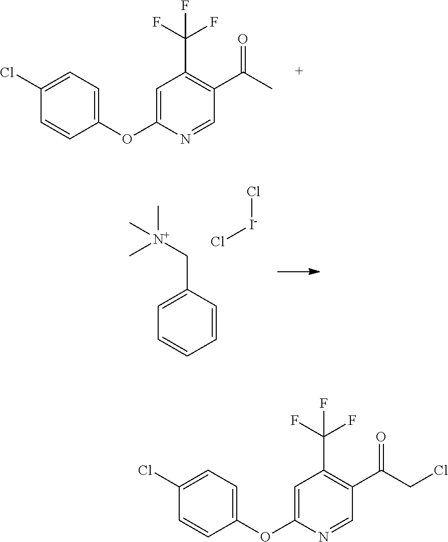

A mixture of 1-[6-(4-chlorophenoxy)-4-(trifluoromethyl)-3-pyridyl]ethanone (3.6 g, 11.4 mmol) and benzyltrimethylammonium dichloroiodate (7.93 g, 22.8 mmol) in 1,2-dichloroethane (30 mL) and methanol (10 mL) was heated to 75° C. for 4 h, before the mixture was concentrated, then diluted with ethyl acetate, washed with Na₂S₂O₃ (10% w/w aqueous solution), washed with brine, dried (over MgSO₄), concentrated, and passed over a plug of silica (heptane/ethyl acetate=85/15, 254 nm) to give 3.4 g (58% yield, 69% pure) of the target compound as a colourless oil, which was used without further purification.

MS (ESI): 348.99 ([M+H]⁺)

### Preparation of Compounds of the Formula (V) According to Process D

### Preparation of 1-[6-(4-chlorophenoxy)-2-(trifluoromethyl)-3-pyridyl]ethanone

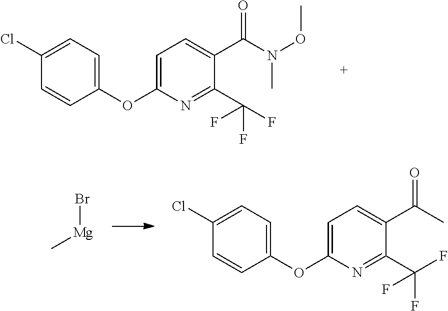

A solution of 6-(4-chlorophenoxy)-N-methoxy-N-methyl-2-(trifluoromethyl)pyridine-3-carboxamide (11.5 g, 31.9 mmol) in THF (150 mL) at 5° C. was treated with methylmagnesium bromide (21.2 mL, 63.7 mmol, 3M solution in diethyl ether). The mixture was then warmed to rt and stirring continued for 4 h at rt, before the reaction was quenched with water, NH₄Cl (saturated aqueous solution), extracted with dichloromethane, dried (over Na₂SO₄), and concentrated to give 8.60 g (82% yield, 96% pure) of the target compound as a pale yellow solid, which was used without further purification.

MS (ESI): 315.03 ([M+H]⁺)

### Preparation of 1-[6-(4-chlorophenoxy)-4-(trifluoromethyl)-3-pyridyl]ethanone

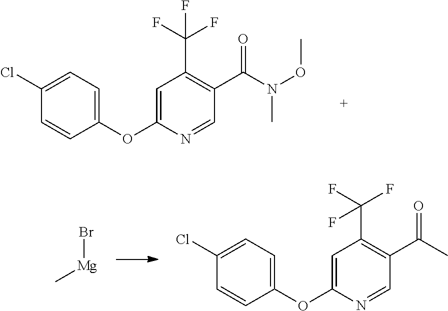

A solution of 6-(4-chhlorophenoxy)-N-methoxy-N-methyl-4-(trifluoromethyl)pyridine-3-carboxamide (6.9 g, 17.4 mmol) in THF (100 mL) at 5° C. was treated with methylmagnesium bromide (11.6 mL, 34.8 mmol, 3M solution in diethyl ether). The mixture was then warmed to rt and stirring continued for 4 h at rt, before the reaction was quenched with water, NH₄Cl (saturated aqueous solution), extracted with dichloromethane, dried (over Na₂SO₄), and concentrated. Flash column chromatography (gradient, up to heptane/ethyl acetate=80/20, 254 nm) gave 3.60 g (61% yield, 94% pure) of the target compound as a colourless oil.

MS (ESI): 315.03 ([M+H]⁺)

### Preparation of Compounds of the Formula (XVI) According to Process D

### Preparation of 6-(4-chlorophenoxy)-N-methoxy-N-methyl-2-(trifluoromethyl)pyridine-3-carboxamide

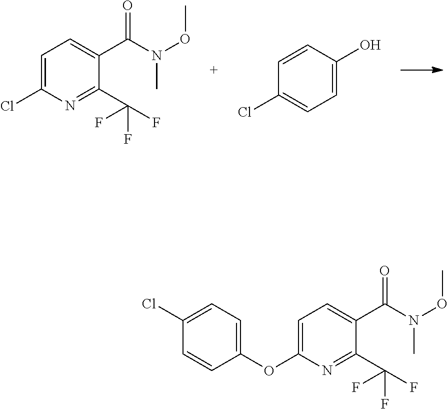 [SMILES: CON(C)C(=O)c1ccc(Cl)nc1C(F)(F)F ; Oc1ccc(Cl)cc1 ; CON(C)C(=O)c1ccc(Oc2ccc(Cl)cc2)nc1C(F)(F)F]

A mixture of 6-chloro-N-methoxy-N-methyl-2-(trifluoromethyl)pyridine-3-carboxamide (9.0 g, 33.5 mmol), 4-chlorophenol (4.3 g, 33.5 mmol), potassium carbonate (11.5 g, 83.7 mmol), copper(I) iodide (638 mg, 3.35 mmol), and N,N,N′,N′-tetramethylethylenediamine (TMEDA; 1.0 mL, 6.7 mmol) in dimethyl sulfoxide (DMSO; 150 mL) was heated for 3 h at 100° C. The reaction mixture was then cooled to rt, water added, extracted with ethyl acetate, dried (over Na₂SO₄), concentrated and passed over a plug of silica (heptane/ethyl acetate=1/1, 254 nm) to give 7.7 g (58% yield, 91% pure) of the target compound as a yellow oil.

MS (ESI): 360.05 ([M+H]⁺)

### Preparation of 6-(4-chlorophenoxy)-N-methoxy-N-methyl-4-(trifluoromethyl)pyridine-3-carboxamide

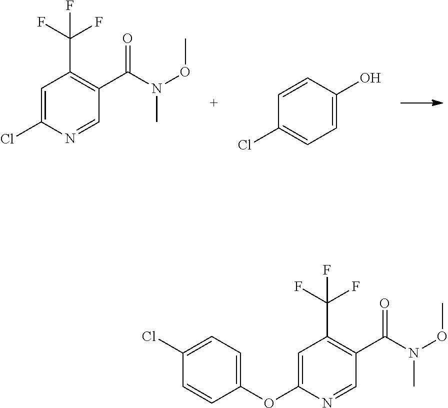 [SMILES: CON(C)C(=O)c1cnc(Cl)cc1C(F)(F)F ; Oc1ccc(Cl)cc1 ; CON(C)C(=O)c1cnc(Oc2ccc(Cl)cc2)cc1C(F)(F)F]

A mixture of 6-chloro-N-methoxy-N-methyl-4-(trifluoromethyl)pyridine-3-carboxamide (5.7 g, 21.3 mmol), 4-chlorophenol (2.7 g, 21.3 mmol), potassium carbonate (7.4 g, 53.3 mmol), copper(I) iodide (406 mg, 2.13 mmol), and TMEDA (0.64 mL, 4.26 mmol) in DMSO (100 mL) was heated for 3 h at 100° C. The reaction mixture was then cooled to rt, water added, extracted with ethyl acetate, dried (over Na₂SO₄), concentrated and passed over a plug of silica (heptane/ethyl acetate=1/1, 254 nm) to give 6.59 g (85% yield, 100% pure) of the target compound as a colourless oil.

MS (ESI): 360.05 ([M+H]⁺)

### Preparation of Compounds of the Formula (I) According to Process B

### Preparation of 1-[6-(4-chlorophenoxy)-2-(trifluoromethyl)-3-pyridyl]-1-cyclopropyl-2-(1,2,4-triazol-1-yl)ethanol according to Process B

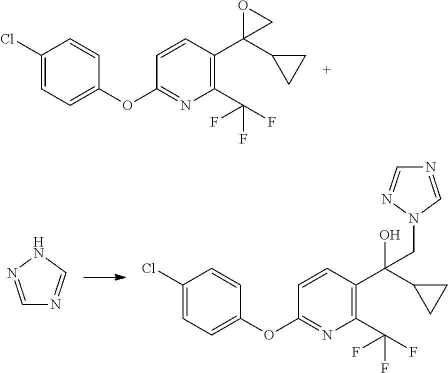 [SMILES: FC(F)(F)c1nc(Oc2ccc(Cl)cc2)ccc1C1(C2CC2)CO1 ; c1nc[nH]n1 ; OC(Cn1cncn1)(c1ccc(Oc2ccc(Cl)cc2)nc1C(F)(F)F)C1CC1]

Epoxide IX.07 (1.0 g, 2.81 mmol), 1H-1,2,4-triazole (194 mg, 2.81 mmol), sodium hydroxide (40 mg, 0.984 mmol), 0.013 mL water in DMF (10 mL) was heated at 120° C. for 22 h, before water, NH₄Cl (saturated aqueous solution) and CH₂Cl₂ were added. Phases were separated, the aqueous phase was extracted twice with CH₂Cl₂, the combined organic extracts were dried over Na₂SO₄ and concentrated to give, after purification via preparative HPLC, the desired alcohol 1.91 (362 mg, 30%) as a colorless oil.

MS (ESI): 425.09 ([M+H]+)

### Preparation of 6-(4-chlorophenoxy)-3-(2-cyclopropyloxiran-2-yl)-2-(trifluoromethyl)pyridine according to Process B

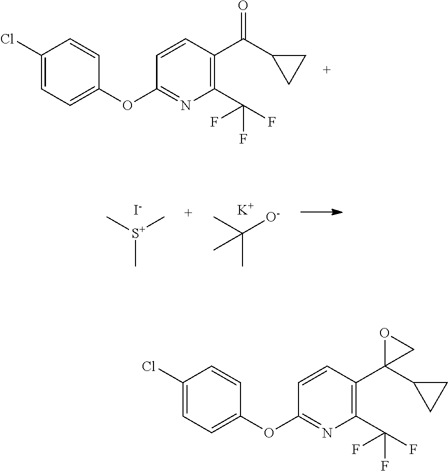

To a suspension of trimethylsulfonium iodide (3.1 g, 15.2 mmol) in THF (100 mL) at 0° C. was added potassium tert-butoxide (1.7 g, 15.2 mmol) in one portion, and the mixture was stirred for 5 min. Then, ketone V.41 (4.0 g, 11.7 mmol) in THF (10 mL) was added, the mixture was warmed to room temperature and stirred for 1.5 h. Then water and CH₂Cl₂ were added, the aqueous phase was extracted with CH₂Cl₂, the combined organic extracts were dried over Na₂SO₄ and concentrated, to give, after flash column chromatography, the desired epoxide IX.07 (138 mg, 3%) as a colorless oil.

MS (ESI): 356.06 ([M+H]+)

### Preparation of [6-(4-chlorophenoxy)-2-(trifluoromethyl)-3-pyridyl]-cyclopropyl-methanone according to Process B

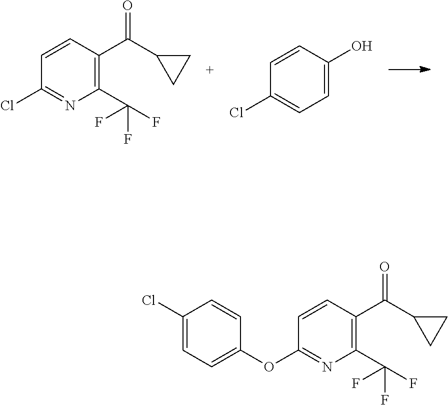 [SMILES: O=C(c1ccc(Cl)nc1C(F)(F)F)C1CC1 ; Oc1ccc(Cl)cc1 ; O=C(c1ccc(Oc2ccc(Cl)cc2)nc1C(F)(F)F)C1CC1]

A mixture of [6-chloro-2-(trifluoromethyl)pyridin-3-yl](cyclopropyl)methanone (6.0 g, 24.0 mmol), 4-chlorophenol (3.1 g, 24.0 mmol), potassium carbonate (8.3 g, 60.1 mmol), copper(I) iodide (458 mg, 2.40 mmol), and N,N,N′,N′-tetramethylethylenediamine (TMEDA; 0.7 mL, 4.8 mmol) in dimethyl sulfoxide (DMSO; 100 mL) was heated for 2 h at 100° C. The reaction mixture was then cooled to rt, water added, extracted with ethyl acetate, dried (over Na₂SO₄), concentrated and passed over a plug of silica (heptane/ethyl acetate=1/1, 254 nm), and recrystallized from CH₂Cl₂ and diisopropyl ether to give 4.2 g (48% yield, 95% pure) of the target compound V.41 as a colorless solid.

MS (ESI): 342.04 ([M+H]+)

### Preparation of [6-chloro-2-(trifluoromethyl)-3-pyridyl]-cyclopropyl-methanone

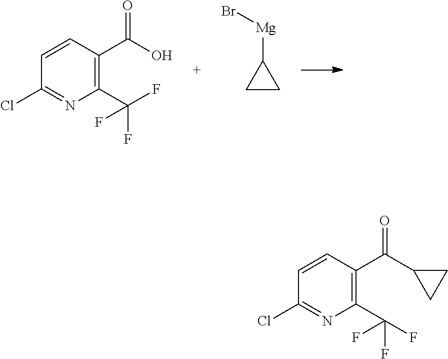

A solution of 6-chloro-2-(trifluoromethyl)pyridine-3-carboxylic acid (6.0 g, 26.6 mmol), thionyl chloride (3.9 mL, 53.2 mmol) and few drops of dimethylformamide in dichloroethane (100 mL) was heated at 85° C. for 4 h, before the mixture was cooled to rt and concentrated. Then dry THF (150 mL) and Fe(acac)₃ (470 mg, 1.33 mmol) were added and the solution was cooled to −78° C., before a solution of cyclopropylmagnesium bromide (69 mL, 0.5 M, 34.6 mmol) was added dropwise, keeping the internal temperature below −70° C. After complete addition, the cooling bath was removed and the reaction was allowed to warm to room temperature. The reaction was then quenched with NH₄Cl (saturated aqueous solution) and extracted with CH₂Cl₂, dried over Na₂SO₄ and concentrated. The target compound [6-chloro-2-(trifluoromethyl)pyridin-3-yl](cyclopropyl)methanone (6.0 g, 87% yield) was used in the following step without further purification.

MS (ESI): 250.02 ([M+H]+)

The following tables illustrate in a non-limiting manner examples of compounds according to the invention as well as intermediates thereof. These compounds have been synthesized according to the synthesis routes described above.

TABLE 1
Compounds according to formula (I-S)
(I-S)

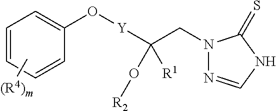

| Ex N° | Y | n | R³ | R | R¹ | R² | m | R⁴ | LogP |
|---|---|---|---|---|---|---|---|---|---|
| I-S-01 | 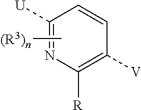 | 0 | — | CF₃ | CH₃ | H | 1 | 4-Br | 3.21 ^{[a]} |
| I-S-02 | 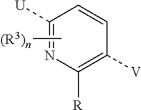 | 0 | — | CF₃ | CH₃ | H | 0 | — | 2.66 ^{[a]} |
| I-S-03 | 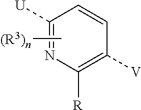 | 0 | — | CF₃ | CH₃ | H | 1 | 4-Cl | 3.11 ^{[a]} |
| I-S-04 | 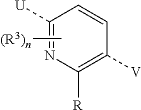 | 0 | — | CF₃ | cyclopropyl | H | 1 | 4-Br | 3.87 ^{[a]} |
| I-S-05 | 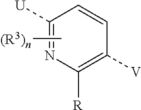 | 0 | — | CF₃ | CH₃ | H | 1 | 4-CF₃ | 3.18 ^{[a]} |
| I-S-06 | 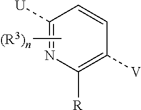 | 0 | — | CF₃ | cyclopropyl | H | 0 | — | 3.27 ^{[a]} |

Log P values:

Measurement of Log P values was performed according to EEC directive 79/831 Annex V.A8 by HPLC (High Performance Liquid Chromatography) on reversed phase columns with the following methods:
^{[a]} Log P value is determined by measurement of LC-UV, in an acidic range, with 0.1% formic acid in water and acetonitrile as eluent (linear gradient from 10% acetonitrile to 95% acetonitrile).
^{[b]} Log P value is determined by measurement of LC-UV, in a neutral range, with 0.001 molar ammonium acetate solution in water and acetonitrile as eluent (linear gradient from 10% acetonitrile to 95% acetonitrile).
^{[c]} Log P value is determined by measurement of LC-UV, in an acidic range, with 0.1% phosphoric acid and acetonitrile as eluent (linear gradient from 10% acetonitrile to 95% acetonitrile).

If more than one Log P value is available within the same method, all the values are given and separated by “+”.

Calibration was done with straight-chain alkan2-ones (with 3 to 16 carbon atoms) with known Log P values (measurement of Log P values using retention times with linear interpolation between successive alkanones). Lambda-max-values were determined using UV-spectra from 200 nm to 400 nm and the peak values of the chromatographic signals.

NMR-Peak lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within patent applications” of the Research Disclosure Database Number 564025.

I-S-01: ¹H-NMR(300.2 MHz, d₆-DMSO):
δ = 13.4298 (2.5); 8.3095 (14.3); 7.8529 (3.6); 7.8238 (4.0); 7.6596 (1.0); 7.6485 (9.8); 7.6413 (3.2); 7.6261
(3.4); 7.6188 (11.0); 7.6078 (1.2); 7.1950 (6.1); 7.1889 (12.0); 7.1816 (3.8); 7.1662 (7.8); 7.1592 (10.2); 7.1481
(1.1); 5.7176 (11.6); 4.5593 (2.7); 4.5136 (4.2); 4.3915 (4.5); 4.3458 (2.9); 3.3500 (11.4); 2.5338 (2.3); 2.5279
(4.8); 2.5218 (6.5); 2.5157 (4.7); 2.5098 (2.2); 2.0944 (6.6); 1.6900 (16.0); 1.6369 (0.4); 0.0180 (3.8)
I-S-02: ¹H-NMR(300.2 MHz, CDCl₃):
δ = 8.1013 (3.2); 8.0719 (3.4); 7.8101 (5.1); 7.4533 (2.6); 7.4469 (1.3); 7.4280 (5.4); 7.4009 (4.4); 7.3055 (1.3);
7.2764 (2.1); 7.2519 (3.3); 7.2263 (2.7); 7.2208 (5.4); 7.2169 (6.2); 7.1917 (4.8); 6.9820 (4.0); 6.9527 (3.9);
5.3272 (3.7); 4.8974 (2.6); 4.8492 (3.3); 4.5615 (3.4); 4.5131 (2.7); 1.7333 (16.0); 1.7080 (0.6); 1.6845 (0.6);
1.2988 (0.6); 1.2765 (0.5); 0.0374 (1.1)
I-S-03: ¹H-NMR(300.2 MHz, CDCl₃):
δ = 11.6984 (0.6); 8.1558 (3.0); 8.1265 (3.2); 7.7995 (8.7); 7.4180 (0.7); 7.4073 (6.1); 7.4001 (2.2); 7.3849 (2.5);
7.3776 (8.1); 7.3669 (1.0); 7.2988 (14.9); 7.2082 (1.0); 7.1976 (8.0); 7.1903 (2.5); 7.1750 (2.2); 7.1679 (6.0);
7.1571 (0.6); 7.0520 (3.7); 7.0228 (3.5); 5.3375 (3.2); 4.9178 (2.7); 4.8695 (3.5); 4.7481 (4.0); 4.5717 (3.8);
4.5233 (3.0); 3.8215 (0.4); 3.7996 (0.9); 3.7778 (0.4); 2.0478 (1.1); 1.9227 (0.4); 1.9120 (0.4); 1.9010 (1.0);
1.8895 (0.4); 1.8791 (0.4); 1.7352 (16.0); 1.6968 (7.0); 0.1065 (4.6); 0.0471 (0.7); 0.0364 (14.0); 0.0255 (0.6)
I-S-04: ¹H-NMR(300.2 MHz, d₆-DMSO):
δ = 13.5521 (1.3); 8.3204 (11.0); 8.3148 (2.3); 8.2024 (3.0); 8.1730 (3.2); 8.0010 (0.3); 7.9725 (0.4); 7.6626
(0.9); 7.6516 (8.2); 7.6444 (2.7); 7.6291 (2.8); 7.6218 (9.4); 7.6109 (1.0); 7.2466 (4.2); 7.2177 (4.8); 7.2077
(9.8); 7.2004 (3.0); 7.1852 (2.7); 7.1779 (8.3); 7.1669 (0.8); 6.8887 (0.5); 6.8594 (0.5); 5.2347 (7.4); 5.1129
(0.9); 4.6564 (0.4); 4.6081 (7.4); 4.5592 (0.4); 3.3402 (16.0); 2.5345 (4.9); 2.5285 (10.2); 2.5224 (13.9); 2.5163
(10.1); 2.5104 (4.9); 1.7887 (0.3); 1.7703 (0.7); 1.7612 (0.9); 1.7442 (1.3); 1.7269 (0.9); 1.7175 (0.8); 1.6994
(0.4); 1.5717 (8.4); 0.6867 (0.8); 0.6712 (1.2); 0.6549 (1.3); 0.6411 (1.0); 0.6254 (0.5); 0.4123 (0.9); 0.3939
(1.1); 0.3834 (1.2); 0.3649 (0.9); 0.3551 (1.1); 0.3450 (1.0); 0.3271 (1.4); 0.3161 (1.3); 0.2995 (1.1); 0.2819
(0.6); 0.2703 (0.4); 0.2408 (0.5); 0.2227 (1.0); 0.2116 (1.4); 0.1925 (1.4); 0.1805 (0.8); 0.0313 (0.6); 0.0204
(14.2); 0.0095 (0.6); −0.0444 (0.3)
I-S-05: ¹H-NMR(300.2 MHz, d₆-DMSO):
δ = 13.4289 (1.1); 8.3178 (7.9); 7.8823 (2.0); 7.8530 (2.7); 7.8450 (3.4); 7.8163 (3.5); 7.4130 (3.4); 7.3850 (3.0);
7.2874 (2.8); 7.2584 (2.6); 5.7623 (6.4); 4.5789 (1.5); 4.5332 (2.3); 4.3911 (2.4); 4.3453 (1.7); 3.3483 (16.0);
2.5343 (2.3); 2.5283 (4.9); 2.5222 (6.7); 2.5162 (4.9); 2.5102 (2.3); 2.0953 (1.4); 1.7106 (8.8); 0.0193 (8.6)
I-S-06: ¹H-NMR(499.9 MHz, d₆-DMSO):
δ = 8.1519 (1.9); 8.1343 (2.0); 7.3149 (1.9); 7.3112 (1.2); 7.2997 (3.2); 7.2979 (3.7); 7.2867 (1.0); 7.2830 (2.7);
7.2187 (3.3); 7.1136 (1.2); 7.1116 (1.0); 7.0988 (2.0); 7.0839 (0.9); 7.0820 (0.7); 7.0559 (2.9); 7.0538 (3.9);
7.0386 (3.2); 7.0368 (3.1); 6.9947 (2.4); 6.9772 (2.4); 6.6172 (1.7); 4.5602 (1.7); 4.5317 (2.3); 4.3668 (2.2);
4.3383 (1.7); 3.8960 (0.6); 3.8817 (0.6); 2.3666 (11.2); 2.3630 (16.0); 2.3595 (13.5); 1.8533 (0.3); 1.8487 (2.5);
1.7627 (0.8); 1.4058 (1.7); 1.3737 (0.5); 1.3685 (0.7); 1.3577 (1.0); 1.3478 (0.7); 1.3425 (0.6); 1.0482 (0.7);
1.0340 (1.3); 1.0198 (0.7); 0.3853 (0.6); 0.3775 (0.9); 0.3676 (0.9); 0.3589 (0.6); 0.3492 (0.3); 0.1066 (0.6);
0.1048 (0.6); 0.0998 (0.6); 0.0950 (0.8); 0.0906 (0.8); 0.0876 (0.8); 0.0783 (0.5); 0.0708 (0.4); 0.0277 (0.5);
0.0245 (0.5); 0.0170 (0.8); 0.0112 (1.2); −0.0002 (1.4); −0.0068 (1.2); −0.0183 (1.1); −0.0251 (0.5); −0.0303 (0.5)

### USE EXAMPLES

### Example A: In Vivo Preventive Test on Botrytis Cinerea (Grey Mould)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of gherkin were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Botrytis cinerea spores. The contaminated gherkin plants were incubated for 4 to 5 days at 17° C. and at 90% relative humidity.

The test was evaluated 4 to 5 days after the inoculation. 0% means an efficacy which corresponds to that of the control plants while an efficacy of 100% means that no disease was observed.

In this test the following compounds according to the invention showed efficacy between 90% and 100% at a concentration of 500 ppm of active ingredient: I-S-01; I-S-02; I-S-03; I-S-05; I-S-06.

### Example B: In Vivo Preventive Test on Puccinia recondita (Brown Rust on Wheat)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of wheat were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Puccinia recondita spores. The contaminated wheat plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test the following compounds according to the invention showed efficacy between 90% and 100% at a concentration of 500 ppm of active ingredient: I-S-01; I-S-03; I-S-04; I-S-05; I-S-06.

### Example C: In Vivo Preventive Test on Septoria tritici (Leaf Spot on Wheat)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of wheat were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Septoria tritici spores. The contaminated wheat plants were incubated for 72 hours at 18° C. and at 100% relative humidity and then for 21 days at 20° C. and at 90% relative humidity.

The test was evaluated 24 days after the inoculation. 0% means an efficacy which corresponds to that of the control plants while an efficacy of 100% means that no disease was observed.

In this test the following compounds according to the invention showed efficacy between 80% and 89% at a concentration of 500 ppm of active ingredient: I-S-06.

In this test the following compounds according to the invention showed efficacy between 90% and 100% at a concentration of 500 ppm of active ingredient: I-S-01; I-S-02; I-S-03; I-S-04; I-S-05.

### Example D: In Vivo Preventive Test on Sphaerotheca fuliginea (Powdery Mildew on Cucurbits)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of gherkin were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Sphaerotheca fuliginea spores. The contaminated gherkin plants were incubated for 72 hours at 18° C. and at 100% relative humidity and then for 12 days at 20° C. and at 70-80% relative humidity.

The test was evaluated 15 days after the inoculation. 0% means an efficacy which corresponds to that of the control plants while an efficacy of 100% means that no disease was observed.

In this test the following compounds according to the invention showed efficacy between 90% and 100% at a concentration of 500 ppm of active ingredient: I-S-01; I-S-02; I-S-03; I-S-04; I-S-05; I-S-06.

### Example E: In Vivo Preventive Test on Uromyces Appendiculatus (Bean Rust)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of bean were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Uromyces appendiculatus spores. The contaminated bean plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-S-06.

In this test the following compounds according to the invention showed efficacy between 90% and 100% at a concentration of 500 ppm of active ingredient: I-S-01; I-S-03; I-S-04; I-S-05.

### Example F: In Vivo Preventive Test on Phakopsora Test (Soybeans)

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous spore suspension of the causal agent of soybean rust (Phakopsora pachyrhizi) and stay for 24 h without light in an incubation cabinet at approximately 24° C. and a relative atmospheric humidity of 95%.

The plants remained in the incubation cabinet at approximately 24° C. and a relative atmospheric humidity of approximately 80% and a day/night interval of 12 h.

The test was evaluated 7 days after the inoculation. 0% means an efficacy which corresponds to that of the untreated control, while an efficacy of 100% means that no disease is observed.

In this test the following compounds according to the invention showed efficacy between 90% and 100% at a concentration of 100 ppm of active ingredient: I-S-01; I-S-03.

### Example G: In Vivo Preventive Test on Venturia Test (Apples)

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous conidia suspension of the causal agent of apple scab (Venturia inaequalis) and then remained for 1 day in an incubation cabinet at approximately 20° C. and a relative atmospheric humidity of 100%.

The plants were then placed in a greenhouse at approximately 21° C. and a relative atmospheric humidity of approximately 90%.

The test was evaluated 10 days after the inoculation. 0% means an efficacy which corresponds to that of the untreated control, while an efficacy of 100% means that no disease is observed.

In this test the following compounds according to the invention showed efficacy between 90% and 100% at a concentration of 100 ppm of active ingredient: I-S-01; I-S-03; I-S-05.

### Example H: In Vivo Preventive Blumeria Test (Barley)

| | | |
|---|---|---|
| Solvent: | 49 | parts by weight of N,N-dimethylacetamide |
| Emulsifier: | 1 | part by weight of alkylaryl polyglycol ether |

To produce a suitable preparation of active compound, 1 part by weight of active compound or active compound combination was mixed with the stated amounts of solvent and emulsifier, and the concentrate was diluted with water to the desired concentration.

To test for preventive activity, young plants were sprayed with the preparation of active compound or active compound combination at the stated rate of application.

After the spray coating had been dried, the plants were dusted with spores of Blumeria graminis f. sp. hordei.

The plants were placed in the greenhouse at a temperature of approximately 18° C. and a relative atmospheric humidity of approximately 80% to promote the development of mildew pustules.

The test was evaluated 7 days after the inoculation. 0% means an efficacy which corresponds to that of the untreated control, while an efficacy of 100% means that no disease is observed.

In this test the following compounds according to the invention showed efficacy between 90% and 100% at a concentration of 500 ppm of active ingredient: I-S-01; I-S-03; I-S-04; I-S-05.

### Example I: In Vivo Preventive Test on Colletotrichum lindemuthianum (Leaf Spot on Bean)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of bean were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Colletotrichum lindemuthianum spores. The contaminated bean plants were incubated for 24 hours at 20° C. and at 100% relative humidity and then for 6 days at 20° C. and at 90% relative humidity.

The test was evaluated 7 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-S-06.

In this test the following compounds according to the invention showed efficacy between 80% and 89% at a concentration of 500 ppm of active ingredient: I-S-03.

In this test the following compounds according to the invention showed efficacy between 90% and 100% at a concentration of 500 ppm of active ingredient: I-S-04; I-S-05.

### Example J: In Vivo Preventive Test on Alternaria brassicae (Leaf Spot on Radish or Cabbage)

| | | |
|---|---|---|
| Solvent: | 5% | by volume of dimethyl sulfoxide |
| | 10% | by volume of acetone |
| Emulsifier: | 1 μl | of Tween ® 80 per mg of active ingredient |

The active ingredients were made soluble and homogenized in a mixture of dimethyl sulfoxide/acetone//Tween® 80 and then diluted in water to the desired concentration.

The young plants of radish or cabbage were treated by spraying the active ingredient prepared as described above. Control plants were treated only with an aqueous solution of acetone/dimethyl sulfoxide/Tween® 80.

After 24 hours, the plants were contaminated by spraying the leaves with an aqueous suspension of Alternaria brassicae spores. The contaminated radish or cabbage plants were incubated for 6 days at 20° C. and at 100% relative humidity.

The test was evaluated 6 days after the inoculation. 0% means an efficacy which corresponds to that of the control plants while an efficacy of 100% means that no disease was observed.

In this test the following compounds according to the invention showed efficacy between 80% and 89% at a concentration of 500 ppm of active ingredient: I-S-04.

In this test the following compounds according to the invention showed efficacy between 90% and 100% at a concentration of 500 ppm of active ingredient: I-S-03; I-S-05.

1. Triazole derivative of formula (I-S) 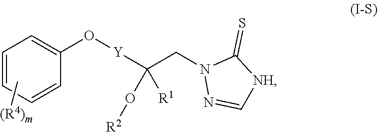
wherein
R¹ represents hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, phenyl, phenyl-C₁-C₄-alkyl, phenyl-C₂-C₄-alkenyl or phenyl-C₂-C₄-alkynyl;
R² represents hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, phenyl, phenyl-C₁-C₄-alkyl, phenyl-C₂-C₄-alkenyl or phenyl-C₂-C₄-alkynyl;
wherein the aliphatic moieties, excluding cycloalkyl moieties, of R¹ and/or R² may carry 1, 2, 3 or up to the maximum possible number of identical or different groups R^{a} which independently of one another are selected from halogen, CN, nitro, phenyl, C₁-C₄-alkoxy and C₁-C₄-halogenalkoxy, wherein the phenyl may be substituted by 1, 2, 3, 4 or 5 substituents which independently of one another are selected from halogen, CN, nitro, C₁-C₄-alkyl, C₁-C₄-alkoxy, C₁-C₄-halogenalkyl, C₁-C₄-halogenalkoxy,
and wherein the cycloalkyl and/or phenyl moieties of R¹ and/or R² may carry 1, 2, 3, 4, 5 or up to the maximum number of identical or different groups R^{b} which independently of one another are selected from halogen, CN, nitro, C₁-C₄-alkyl, C₁-C₄-alkoxy, C₁-C₄-halogenalkyl and C₁-C₄-halogenalkoxy;
each R⁴ represents independently from each other halogen, CN, nitro, C₁-C₄-alkyl, C₁-C₄-halogenalkyl, C₃-C₆-cycloalkyl, C₃-C₆-halogencycloalkyl, C₁-C₄-alkyl-C₃-C₆-cycloalkyl, C₁-C₄-alkoxy, C₁-C₄-halogenalkoxy, hydroxy-substituted C₁-C₄-alkyl, C₂-C₆-alkenyl, C₂-C₆-halogenalkenyl, C₂-C₆-alkynyl, C₂-C₆-halogenalkynyl, C₁-C₄-alkylsulfanyl, C₁-C₄-halogenalkylsulfanyl, formyl, C₁-C₄-alkylcarbonyl, pentafluoro-λ⁶-sulfanyl or —C(R^{4a})═N—OR^{4b}, wherein R^{4a} and R^{4b} represent independently from each other hydrogen, C₁-C₆-alkyl or phenyl;
m is an integer and is 0, 1, 2, 3, 4 or 5;
Y represents a 6-membered aromatic heterocycle containing 1 or 2 nitrogen atom(s) as heteroatom(s) selected from 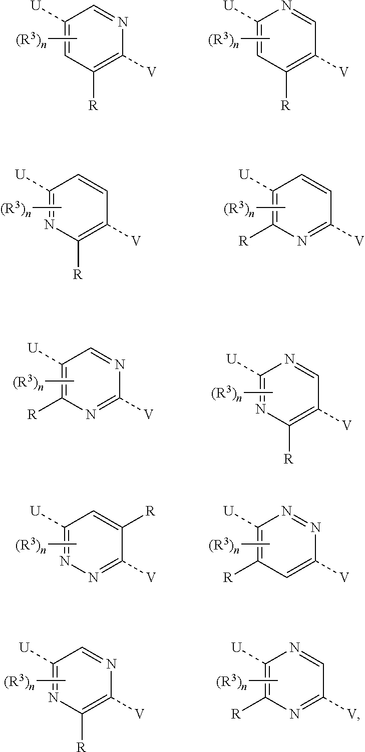
wherein Y is connected to the O of formula (I-S) via the bonds identified with “U” and Y is connected to the CR¹(OR²) moiety of formula (I-S) via the bonds identified with “V” and
wherein
R represents hydrogen, C₁-C₂-halogenalkyl, C₁-C₂-halogenalkoxy, C₁-C₂-alkylcarbonyl or halogen;
each R³ represents independently from each other halogen, CN, nitro, C₁-C₄-alkyl, C₁-C₄-halogenalkyl, C₁-C₄-alkoxy or C₁-C₄-halogenalkoxy;
n is an integer and is 0 or 1;
and/or a salt or N-oxide thereof.

2. Triazole derivative of formula (I-S) according to claim 1, wherein
R¹ represents hydrogen, methyl, ethyl, propyl, isopropyl, butyl, cyclopropyl, CF₃, benzyl, allyl, CH₂C≡C—CH₃ or CH₂C≡CH,
and/or
R² represents hydrogen
and/or a salt or N-oxide thereof.

3. Triazole derivative of formula (I-S) according to claim 1, wherein
each R⁴ represents independently from each other Br, CI, F, CN, nitro, methyl, ethyl, n-propyl, isopropyl, CHF₂, CF₃, methoxy, OCF₃, SCH₃, SCF₃, pentafluoro-λ⁶-sulfanyl or —C(R^{4a})═N—OR^{4b},
wherein R^{4a} represents hydrogen, methyl, ethyl, n-propyl, isopropyl, n-butyl, isobutyl or tert.-butyl, optionally hydrogen or methyl, R^{4b} represents hydrogen, methyl, ethyl, n-propyl, isopropyl, n-butyl, isobutyl or tert.-butyl, optionally hydrogen or methyl,
and/or
m is 1
and/or a salt or N-oxide thereof.

4. Triazole derivative of formula (I-S) according to claim 1, wherein
Y represents a 6-membered aromatic heterocycle containing 1 or 2 nitrogen atom(s) as heteroatom(s) selected from 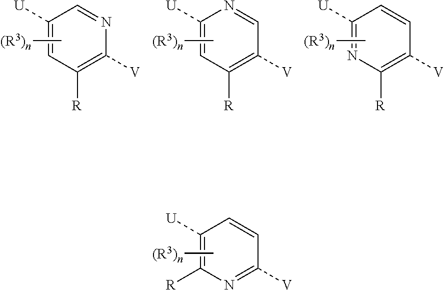
wherein Y is connected to the O of formula (I-S) via the bonds identified with “U” and Y is connected to the CR¹(OR²) moiety of formula (I-S) via the bonds identified with “V” and
wherein R, R³ and n are defined according to claim 1
and/or a salt or N-oxide thereof.

5. Triazole derivative of formula (I-S) according to claim 1, wherein
Y represents a 6-membered aromatic heterocycle containing 1 or 2 nitrogen atom(s) as heteroatom(s) selected from 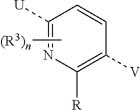
wherein Y is connected to the O of formula (I-S) via the bonds identified with “U” and Y is connected to the CR¹(OR²) moiety of formula (I-S) via the bonds identified with “V” and
and/or a salt or N-oxide thereof.

6. Triazole derivative of formula (I-S) according to claim 1, wherein
R represents CF₃ or Cl,
and/or
n is 0
and/or a salt or N-oxide thereof.

7. Triazole derivative of formula (I-S) according to claim 1, wherein
R¹ represents hydrogen or C₁-C₄-alkyl;
R² represents hydrogen;
R⁴ represents CF₃, CHF₂, OCF₃, Br, Cl or pentafluoro-λ⁶-sulfanyl;
m is 1;
Y represents 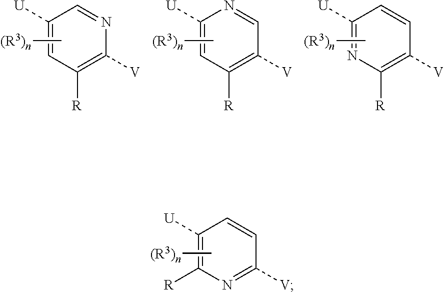
wherein Y is connected to the O of formula (I-S) via the bonds identified with “U” and Y is connected to the CR¹(OR²) moiety of formula (I-S) via the bonds identified with “V” and
R represents C₁-halogenalkyl, F or Cl;
n is 0;
and/or a salt or N-oxide thereof.

8. Triazole derivative of formula (I-S) according to claim 1, wherein
R¹ represents hydrogen or C₁-C₄-alkyl;
R² represents hydrogen;
m is 0;
Y represents 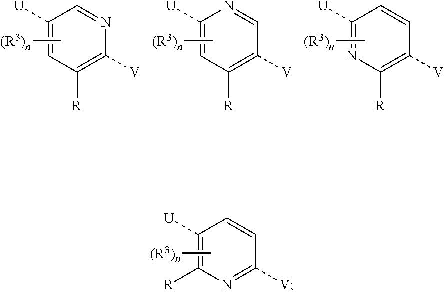
wherein Y is connected to the O of formula (I-S) via the bonds identified with “U” and Y is connected to the CR¹(OR²) moiety of formula (I-S) via the bonds identified with “V” and
R represents C₁-halogenalkyl, F or Cl;
n is 0;
and/or a salt or N-oxide thereof.

9. Triazole derivative of formula (I-S) according to claim 7, wherein
R¹ represents hydrogen or methyl;
Y represents 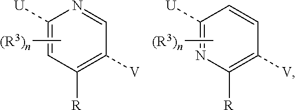
optionally 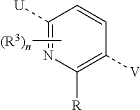
wherein Y is connected to the O of formula (I-S) via the bonds identified with “U” and Y is connected to the CR¹(OR²) moiety of formula (I-S) via the bonds identified with “V” and
R represents CF₃ or Cl;
n is 0;
and/or a salt or N-oxide thereof.

10. Composition for controlling one or more harmful microorganisms, optionally for controlling phytopathogenic harmful fungi, comprising at least one compound according to claim 1, and at least one extender and/or surfactant.

11. Composition according to claim 10 comprising at least one further active ingredient selected from the group of insecticides, attractants, sterilants, bactericides, acaricides, nematicides, fungicides, growth regulators, herbicides, fertilizers, safeners and semiochemicals.

12. Method for controlling one or more harmful microorganisms in crop protection and in protection of one or more materials, comprising applying at least one compound according to claim 1, and/or a composition thereof to the harmful microorganisms and/or a habitat thereof.

13. Method according to claim 12, wherein the harmful microorganism is a phytopathogenic harmful fungus.

14. A product comprising at least one compound according to claim 1, and/or a composition thereof for control of one or more harmful microorganisms, optionally phytopathogenic harmful fungi, in crop protection and/or in protection of one or more materials.

15. A product comprising at least one compound according to claim 1, and/or a composition thereof for treatment of a plant, optionally a transgenic plant, or for treatment of seed, optionally seed of a transgenic plant.
